(19) United States
(12) Patent Application Publication
(10) Pub. No.: US 2006/0204968 A1
(43) Pub. Date: Sep. 14, 2006

(54) Tools for diagnostics, molecular definition and therapy development for chronic inflammatory joint diseases

(76) Inventors: Thomas Haeupl, Erkner (DE); Ute Ungethuem, Berlin (DE); Stefan Blaess, Berlin (DE)

Correspondence Address: JORDAN AND HAMBURG LLP, 122 EAST 42ND STREET, SUITE 4000, NEW YORK, NY, 10168, US

(21) Appl. No.: 10/727,167

(22) Filed: Dec. 1, 2003

Related U.S. Application Data
(63) Continuation of application No. PCT/DE02/02010, filed on May 30, 2002.

(30) Foreign Application Priority Data
May 30, 2001 (DE) 101 27 572.2

(51) Int. Cl. C12Q 1/68; C12P 19/34; G01N 33/567

(52) U.S. Cl. 435/6; 435/91.2; 435/7.2

(57) ABSTRACT

The invention relates to tools for the diagnosis, molecular definition and development of treatment of chronic inflammatory joint diseases and other inflammatory, infectious or tumourous diseases. According to the invention, genome data (genomics), proteome data (proteomics) and immunome data (immunomics) are used in the analysis and development of treatment of chronic joint diseases. The invention is based on the use of gene sequences and derived mRNAs and proteins, in addition to antibodies having a specific nature for the derived proteins, for characterising inflammatory and non-inflammatory rheumatic joint diseases, auto-immune diseases and infectious diseases. Etiologically significant pathogenicity principles of chronic inflammatory joint diseases which have been unclear until now can be derived from the examinations carried out. Furthermore, interpretation algorithms can be created for the classification, prognosis evaluation and treatment optimisation of said joint diseases, and new strategies for treatment and points of attack for medicaments can be derived.

The invention refers to tools for diagnostics, molecular definition and therapy development for chronic inflammatory joint diseases and other inflammatory, infectious or tumourous diseases. These tools are based on genomic data (Genomics), proteomic data (Proteomics) and immunological data (Immunomics) in the analysis and therapy development for chronic joint diseases. The invention is based both on the use of gene sequences and deduced mRNAs and proteins and on the use of antibodies being specific for the deduced proteins for characterizing inflammatory rheumatoid and non-inflammatory rheumatoid joint diseases, autoimmune diseases and infectious diseases. Starting from the investigations one can derive etiologically important pathogenicity principles of the hitherto unexplained chronic inflammatory joint diseases. Moreover, one can construct interpretation algorithms for the classification, prognostic evaluation and therapy optimization of these joint diseases, and moreover one can draw conclusions for novel therapeutic strategies and therapeutic targets.

### Overview of the Prior Art

Technical Problem

The etiology of chronic inflammatory joint diseases is not yet understood. The rheumatoid arthritis (RA—see list of abbreviations following the examples) is the classic example for these diseases. Major processes of the disease take place in the synovial membrane, which is altered in an inflammatory manner, thereby leading to a chronic joint lesion. The clinical picture observed is very heterogeneous, suggesting, that one is faced with several entities showing the common symptom of destructive synovitis. These diseases also have to be understood as systemic diseases, in which a multitude of changes is observed in the blood and which sometimes result in severe organic manifestations.

Overactive inflammatory activities due to dysregulations in the inflammatory cascade are discussed as major pathogenic mechanisms. Furthermore, autoimmune reactions have been described, which suggest a role of the specific humoral and cell-mediated immune system in the pathogenic process. However, also other mechanisms like enzymatic tissue destruction, cell and tissue proliferation or regeneration are discussed, these factors also potentially playing a crucial role in pathogenesis.

It was so far not possible to finally determine, if these mechanisms of pathogenesis are the sole and exclusively relevant ones. It is furthermore unknown, which parameters are able to simultaneously encompass all these changes. In consequence of the insufficient pathophysiological understanding, numerous therapeutics are available, the major examples of which however only follow one main therapy concept:

Focusing on the common symptom of excessive inflammation, the current therapy thus aims to suppress inflammation. So-called basal therapies display an immunomodulating and disease-modifying character. They interfere with basal mechanisms of cellular metabolism and cellular activity (e.g. Methotrexate, Azathioprine). The comprehensive principles of the molecular mechanism of these therapies in the joint diseases however are incompletely understood. In consequence, there is a lack of respective parameters for controlling the therapeutic efficiency of single basal therapies in a differential and specific manner in the individual case.

Previous Tools

Patients with joint diseases are nowadays evaluated according to the following criteria in the clinical routine: reported progression of the disease (anamnesis), clinical picture (disease pattern observed in the joints, organic manifestation), parameters of inflammation (unspecific inflammatory parameters observed in serum electrophoresis, sedimentation rate, and C-reactive Protein), autoimmunogenic parameters (rheumatoid factor, antinuclear antibodies and a few specific auto-antibodies like anti-Ro, -La, -U1RNP, -Sm, -Histone, -Scl70, -Centromere, -dsDNA, -phospholipid-antibody), genetic predisposition based on HLA-markers (DR4, B27, DR3), image forming (destructive alterations in the X-ray picture of the joints), extended organ diagnostics by means of routine parameters of laboratory diagnostics (liver enzymes, muscle enzymes, kidney retention values) and, if favorable, further techniques of sonography, radiology and magnetic resonance tomography. These only allow for very limited predications concerning the aggressiveness of the disease to be prognosticated or concerning the concrete expectations of success of a basal therapeutic in the individual patient. Moreover, the diagnostic criteria are nowadays not designed for sufficiently classifying the diversity of manifestations in the most common arthritic disease, the RA (1, see references following the examples). Especially in the early phase of the disease, diagnosis is difficult and uncertain. After an endurance of the disease of just one year however, the majority of the patients already suffers from irreversible joint lesions. It is known from early-stage arthritis studies, that a diagnosis being earlier confirmed and followed by an adequate therapy goes along with essential improvements concerning the long-term development of the disease. Novel methods and criteria, integrating molecular features beyond the clinical picture are thus extremely necessary.

Also the progress monitoring of the therapeutic success is hitherto accomplished by means of the above mentioned methods of diagnosis. Many of these parameters only change very slowly. They require many weeks to months of observation in order to come to a conclusion, if the chosen therapeutic is effective. Often the therapeutic has to be changed due to insufficient amelioration and progression of the disease. Healing of the diseases is generally impossible by using the therapeutics currently available.

Experimental Approaches

There exist many experimental approaches beyond the established tools in order to improve the diagnostics especially of RA.

They refer to the search for key proteins, which 1.) maintain or prevent the progression of inflammation in a central position, 2.) are decisively taking part in the enzymatic destruction of the cartilage and bone matrix or which inhibit the responsible enzymes, or 3.) can induce regenerative and reparative processes or inhibit their antagonists. Here for example, the role of the inflammation-mediating cytokines Tumour Necrosis Factor (TNF-) alpha and Interleukin (IL-) 1 beta has proven to be essential and has thus introduced respective therapeutic approaches into clinical use. Although an inhibition of TNF-alpha can in many cases ameliorate a RA being not sufficiently affected by common tools, these positive results however do not lead to a healing of the disease. Partly, the inhibition is such strong, that infections or even septic complications arise and a sufficient control of arthritis is nevertheless not accomplished. This suggests, that the TNF-alpha-mediated pathway of inflammation is at least not the only central pathogenic mechanism of the disease. Besides the two mentioned cytokines, the role of numerous other signal substances in the pathogenesis of arthritis is under investigation. In addition, therapeutic intervention increasingly focuses on the corresponding intracellular signal pathways.

Moreover, the matrix metalloproteinases and cathepsins are in the center of the enzymatic destruction of bone and cartilage.

Investigations of regenerative mechanisms are just at the beginning of research. In the first place one has to mention signal substances belonging to the Transforming Growth Factor (TGF-) beta-family. A large number of them plays a crucial role in the development of the locomotor system. First investigations on synovial tissue and cartilage have shown, that members of this group of growth factors and morphogens are also produced in the adult synovial tissue. For inflammatory joint diseases, we were able to show in our own investigations, that some of these factors obviously show a relative decrease. Furthermore, it was able to be shown for Bone Morphogenetic Protein (BMP-) 7, that the cellular invasion into developing artificial cartilage tissue was suppressed (2).

Many of the mentioned factors and enzymes are also to be found in other joint diseases like osteoarthrosis or the reactive arthritic diseases and therefore—being regarded for themselves—do not constitute a specific diagnostic parameter.

The experimental approaches also focus on the fact, that auto-reactive T- and B cells arise in RA, which is accordingly classified into the group of autoimmune diseases. This classification goes back to the discovery of the so-called rheumatoid factor, an auto-antibody, which is directed against immunoglobulin G. Rheumatoid factors however only occur in about two thirds of the RA-patients, but are also present in other rheumatoid and non-rheumatoid diseases and even in up to 5% of the healthy population (even to a higher degree with increasing age). The occurrence of rheumatoid factors seemingly is a physiological reaction of the body under certain pathological conditions, like e.g. the bacterial endocarditis. Auto-reactive B cells with a specificity for IgG are seemingly present in a major part of the population and can be activated by different mechanisms. The term “rheumatoid factor” was nevertheless maintained, since it only offers a diagnostic and prognostic meaning for RA.

The same characteristics however do also qualitatively apply for nearly all auto-antibodies, which are hitherto known for RA: the frequency of positive patients is significantly less than 100% and the disease specificity in part is also significantly less than 100%. The pronounced clinical heterogeneity of RA in respect to the disease pattern, the intensity of inflammation and the intermittent character is thus in parallel to a heterogeneity of the immunologically dysregulated processes. This clinical and immunological heterogeneity also supports the speculation, that the “rheumatoid arthritis” may be a general term for different disease entities. A typical example for this is the differentiation between the RF-positive and RF-negative (RF—rheumatoid factors) RA, whereat the first is said to have a more severe progression with a higher destructive potential and a systemic humoral activity. The term “seronegative” erroneously implies even the absence of any auto-antibody. However, neither the rheumatoid factor nor anyone of the other known autoreactivities could be confirmed as an etiological cause for the rise of RA or one of its postulated subforms or progress forms.

Auto-antibodies are used for diagnostic classification in case of other rheumatoid autoimmune diseases like the collagenoses with systemic Lupus erythematodes (SLE) as their major member. A primary pathogenicity of these auto-antibodies is constantly and repeatedly discussed. It is certain, that a high titer of auto-antibodies in combination with an unscheduled, excessive release of auto-antigens during an intermittent episode of the disease and the subsequent formation of immune complexes and complement activation is associated with organic lesions, especially of the kidney, and with vasculitic features. The role of the auto-reactive B- and T cells in RA however is not determined. Instead, novel auto-antigens are evermore described as targets of an autoreactive immune response in RA. Some of these antigens are well characterized in respect of their biochemistry and antigenic features, others however are only understood in respect of a few parameters. Some of these auto-antibodies were very promising for their discoverers, since the B and/or T cell-response appeared to be highly specific for RA. The interest in these antibodies however always quickly vanished, when the same autoreactivities were also detected in other autoimmune diseases. Meanwhile, numerous T cell-associated autoreactivities have been discovered for RA, only a very few of which however are specific for RA.

Heat Shock Proteins

The RA has soon been suspected to constitute an infectious disease. Therefore, a diversity of xenogenous antigen sources—in most cases of microbial or viral origin—was investigated in order to detect potential pathogens acting as triggers of autoreactivity. One of the potential RA-inducing agents was Mycobacterium tuberculosis, since in the animal model it induces the adjuvant-arthritis, a disease being similar to human RA in certain aspects. This experimental disease was also able to be induced by the mycobacterial heat shock protein 65 (mt-Hsp65) or by T cells, which are specific for this antigen. Heat shock proteins support native proteins in developing their correct three-dimensional structure, thereby creating tertiary and quatemary structures. mt-Hsp65 is homologous to the essential Hsp60 in mammalian species. Reports about mt-Hsp65-specific T cells and antibodies in the synovial fluid of RA-patients suggested, that the strongly homologous human Hsp60 would be recognized as an antigen in RA-patients. These antibodies however, are not specific for RA: They also occur in patients with Reiter's syndrome, SLE and active tuberculosis, but also in healthy persons.

Although the reactivity against mt-Hsp65 does not seem to play a dominant role in RA, human Hsp60 might nevertheless be important in the pathogenesis of RA: In its amino acid sequence, human Hsp60—in regions of 11 to 22 amino acids—has an identity with proteins like cytokeratin and Hsp90. It is thus conceivable, that autoreactive T cells or antibodies against these proteins originally result from a natively occurring—but strictly regulated—Hsp60-reactivity.

Dna J

Dna J, the bacterial stress protein having homology to mammalian Hsp70, provides the amino acid sequence QKRAA, better known under the designation “Shared Epitope”, which confers predisposition to RA (3). This epitope also occurs in the protein gp110, which is encoded by the Epstein-Barr virus (EBV). Dna J is the target of autoreactive T cells under the conditions of RA, but not in the healthy patient (4). Although it is still unknown, in which way Shared Epitope confers RA-predisposition, one conceivable mechanism may be the generation of the Shared Epitope-peptide from non-MHC-proteins and the subsequent presentation on MHC class II-molecules, thereby inducing an immune response against foreign (EBV-gp 110) and self (MHC class II).

EBV-Encoded Nuclear Antigen

Epstein-Barr virus (EBV) has soon been suspected to cause RA, although it has just recently been possible to detect this virus in the synovial fluid of RA-patients. An antibody, directed against the EBV-encoded nuclear antigen (EBNA-1), showed strong reactivity with a p62-protein from synovial mesothelial cells in patients with RA. EBNA-1 contains a glycine-alanine-rich repeat sequence (IR-3), which is recognized by auto-antibodies in patients with RA, SLE, systemic sclerosis (SSc) and infective mononucleosis, but also in healthy individuals in comparable frequency. EBNA-1 shows cross-reactivity with numerous human proteins, typically via the IR-3 sequence. Among these, essential examples are p62 and p542, whereat the latter is mainly recognized by antibodies from patients with infective mononucleosis, but also from RA-patients. P542, due to its high sequence identity with the mouse hnRNP designated “Raly” and similarities with the human hnRNP C2, has recently been identified as the 71 k component of hnRNPs.

Sa-Antigen; Filaggrin, Citrullinated Peptides/Proteins

The Sa-antigen (5) and filaggrin are two recently discovered antigens, which are not present in the inflamed joint, but attracted attention because of the highly RA-specific immune response. The Sa-antigen is a 50 k-protein derived from human spleen and placenta. Sa-specific antibodies occur in 43% of the RA-patients and have a disease specificity of 78% to 99%. Filaggrin is a 42 k-protein, which is responsible for cross-linking intermediary filaments, in particular cytokeratin, and which is present in the endothelium. Filaggrin-specific antibodies are apparently the same as the “antiperinuclear factor”, which was described a long time before, and as the so-called anti-keratin antibodies. The major determinant of the epitope(s) being recognized by the anti-filaggrin antibodies is citrullin, a post-translationally modified arginine (6, 7). The sensitivity of these antibodies is between 36% and 91%, and the specificity is between 66% and 100%. Although filaggrin only occurs extra-articularly, citrullin meanwhile has been successfully detected also in synovial cells.

Collagen II

Collagen type II is a major component of the joint cartilage and thus seems to be predisposed as an auto-antigen for RA. Accordingly many studies have dealt with the role of the collagen-specific immune response. Mouse T cells reacting with the bovine collagen type II are specific for an epitope, which also occurs in human collagen II and which furthermore overlaps with an important T cell epitope from mice suffering from collagen-induced arthritis. Collage type II is a component of the extracellular matrix, which produces triple helices from identical tropocollagen subunits, which themselves are processed from the even larger procollagens. B cells having specificity for collagen seem to occur in the inflamed joints of RA-patients in a more pronounced manner. T cells being specific for Collagen II occur as well in RA-patients as in healthy individuals.

The collagen reactivity attracted particular attention within the scope of the studies of oral tolerance in RA. In the animal model, oral tolerance can be induced by means of antigens occurring in the compartment of the (autoimmune) inflammation, but not being necessarily involved in the inflammatory process themselves. If such an antigen is orally applied, T cells having specificity for the fed antigen are apparently tolerated and are then capable to produce the so-called Bystander-Suppression via suppressive factors, like e.g. IL-10 and TGF-β, in another place, namely the inflamed joint. T cells being such specific for collagen II were intended to downmodulate the inflammation in RA. However, three placebo-verified double blind studies of oral tolerance did not reveal a significant improvement of disease activity, when collagen II was applied. A similar result also applies for clinical studies with peptides from Hsp65 (Subreum).

Chondrocyte Antigen 65 (CH65)

Chondrocyte membranes were reported to be a target of autoreactive T cells in RA- and arthrosis-patients (8), whereas T cells of normal donors did not show such a reaction. Moreover, chondrocyte membranes are recognized by auto-antibodies in 70% of the RA-patients. The respective antigen is the cartilage-specific CH65, which shows a sequence similarity to mycobacterial Hsp65 and certain cytokeratins. CH65 displays a high proportion of glycine, similar like, but not identical with Hsps. Although the sequences are similar to those of keratins, they are nevertheless completely untypical for them. Such similarities allure to arrive at the idea of a molecular mimicry between human/mycobacterial Hsps and other proteins. However, no cross-reactivity has been found between the monoclonal antibodies, which are specific for CH65, cytokeratin or Hsp65. T cell reactivity was just investigated against unpurified chondrocyte membranes.

HC gp39

In the synovial fluid, numerous antigens occur, which were only tested in little groups of patients and controls. One example is the Human Cartilage-Glycoprotein (HC gp39), an important product, which is secreted by articular chondrocytes, synovial cells, macrophages of late stages of differentiation, and by neutrophils. The gp39-level in patients with a degenerative joint disease is increased in the serum and the synovial fluid in comparison to healthy individuals. Later it was shown, that an increased titer not only occurs in case of osteoarthrosis, but also in case of colorectal carcinoma, alcohol-induced liver cirrhosis and breast cancer. gp39 not only has a role in reorganising tissues and degrading the extracellular matrix, but it also is a target of autoreactive T cells in RA. Accordingly, also peptides from the gp39-sequence were tested to bind HLA-DR4 (DRB1*0401) and to stimulate T cells. gp39-reactive T cells were detected in 8 of 18 RA-patients and 3 of 11 healthy individuals. In the animal model, an immunization of Balb/c-mice leads to a chronic arthritis with intermittent episodes, which again was able to be healed by a nasal application of gp39.

Rheumatoid Factor

The best known auto-antigen in RA is at the same time not tissue-specific, but can occur nearly ubiquitously. It is the immune globulin G (IgG) as the target of further antibodies, the so-called rheumatoid factors (RF). The rheumatoid factor is still the only serological parameter, which is comprised within the criteria of the American College of Rheumatology (ACR-criteria). The pathological relevance of RF for RA is still controversially discussed, since RF also occurs in patients with SLE, Sjogren's syndrome, endocarditis, liver diseases and even in healthy persons. The RF-titer is not strictly correlated with the clinical or serological activity of RA or with the degree of joint destruction.

hnRNP A2-Protein (RA33)

The A2-protein belonging to the human nuclear ribonucleoproteins (hnRNPs) is a ubiquitous protein, which was originally described as RA33 auto-antigen. In the following, both its identity with the A2-component and its reactivity with sera from patients suffering from SLE, mixed collagenoses (Mixed Connective Tissue Disease; MCTD) and other diseases, were shown. A2 is present as a complex with numerous other factors, which together represent the hnRNPs in the nucleus. The exact function of A2 is unknown, although a function in splicing the human nuclear ribonucleic acid (hnRNA) is supposed. Accordingly, A2 provides two RNA-binding domains and a nuclear import/export signal. Antibodies in RA and SLE are directed against the region between the RNA-binding domains, whereas those in MCTD-patients (Mixed Connective Tissue Disease) recognize a discontinuous epitope, which is comprised of both RNA-binding domains. It is not yet clear, how the immune system gets into contact with A2. From the view-point of the homunculus however, the hnRNPs are good candidate-antigens for RA. Up to now however, one can only speculate, that A2—under certain circumstances—arrives at the cellular surface, e.g. during the cell decay in the course of an inflammation.

Calpastatin

Calpastatin is a ubiquitous cytoplasmatic protein having a molecular mass of 72 k and four inhibitory domains for calpains. Calpains comprise a family of cysteine-proteases, which are suspected to be involved in the joint destruction in rheumatoid diseases. Calpains occur in the cytoplasm and are stringently regulated by calcium ions for activation and by calpastatin for inhibition. After cell activation, calpastatin occurs also extracellularly und is thus accessible for antibodies. Calpastatin is recognized by auto-antibodies in patient with RA, SLE, polymyositis/dermatomyositis (PM/DM), MCTD, activated arthrosis and venous thrombosis. In the animal model of calpastatin-deficient rats, no symptoms of arthritis are able to be induced. Calpastatin, calpains and calpastatin-specific antibodies are present in the inflamed joints of RA- and OA-patients and might thus be involved in the pathogenesis of these diseases.

Calreticulin

Calreticulin is an ubiquitous protein of the endoplasmatic reticulum (ER), which—under certain circumstances—also occurs in the nucleus, the cytoplasm and on the cellular surface. It constitutes a highly conserved Ca⁺⁺-binding protein. Calreticulin is the target of auto-antibodies in a number of different diseases of auto-immunological or inflammatory origin, mainly in SLE and onchocercosis, but also in RA. Furthermore, the RA-associated haplotype DR4Dw4/DR53 binds a peptide from Calreticulin.

BiP (Heavy Chain Binding Protein)

A further promising target antigen for the homunculus of RA is the ubiquitous BiP (Binding Protein), which was originally described as Heavy Chain Binding Protein, since it interacts with the heavy chains of immunoglobulins. BiP itself is a resident ER-protein and possesses a peptide sequence preventing the protein from being exported under normal conditions. Meanwhile is has been revealed, that BiP is a so-called molecular chaperon, which in this role interacts with most of the proteins, which are introduced into the endoplasmatic reticulum (ER) and enter the secretory pathway. Beyond this essential functional feature, BiP is overexpressed under the effect of stress factors like heavy metal ions or agents affecting the level of calcium ions in the cell or the integrity of protein biosynthesis. Under these conditions it can even be detected within the nucleus, but also on the cellular surface.

BiP is a target of auto-reactive antibodies and T cells in 66% of RA-patients; it was originally described as p68 in the context of RA. The disease specificity of these auto-antibodies is 99% and thus extremely high. The antigen is O-glycosylated and it is supposed, that this modification might have a regulatory function like mono-O-GlcNAc has in many other proteins. In these proteins, the switch from the O-GlcNAc to the O-phosphate-modification is coupled with a change of the state of activation or of the cellular compartment. In a similar manner, a stress-induced shift of BiP from the ER to the nucleus or to the cellular surface might be of pathogenic relevance. The presence of BiP on the cellular surface, which is rather untypical, might serve as a signal of alarm or activation for other cells, and also for cells of the immune system. In RA such an activation may occur by a local infection or by a tissue being otherwise deteriorated by inflammation. In consequence of the cell- or tissue damage, BiP might arrive at the surface of injured cells, where it then becomes a target of auto-reactive T cells. There exist hints, that these BiP-reactive T cells also occur under natural conditions, under which these T cells are then downregulated by regulatory T cells after the inducing conditions have ceased. The regulatory cells are antigen-specific and HLA-restricted. Thereby, the HLA-restriction of regulatory T cells is apparently distinct from the HLA-restriction of effector T cells and allows to be specifically inhibited. In this context, the epitope O-GlcNAc might again have a crucial role: It is well conceivable, that this epitope is not only a target of the auto-antibody response, but also of the T cell response. A further protein, which was isolated from the synovial fluid, the function of which however largely goes beyond this compartment, is the p205-antigen. It is a target of autoreactive T-cells in RA-patients. P205 is also expressed in the synovial membrane and probably constitutes the antigen with the highest T cell stimulating capacity in RA at all, partly reaching the proliferation rate, which can be obtained by means of synovial fluid or even by means of the lectin phytohemagglutinin (PHA). The function of the p205-antigen is still unknown. However, it contains a sequence of 11 amino acids, which is identical with a section from IgG, namely within the region between the constant domains C_{H}2 and C_{H}3, a region, in which the binding of rheumatoid factors takes place. This region of p205 is both bound by monoclonal rheumatoid factors and also recognized by autoreactive T cells. Furthermore, p205-specific T cells, when being stimulated by cognate antigen, have a supportive effect on B cells in the secretion of rheumatoid factors. It thus has to be assumed, that herewith for the first time an antigen has been discovered, which possesses T cell reactivity and is furthermore capable to support IgG-specific B cells in affinity maturation. In contrast to this, a T cell reactivity against intact IgG or IgG-fragments was not able to be found so far. Possibly, the amino acid sequence of p205 might constitute a peptide, which in vivo is not or not sufficiently produced during the processing of IgG. Thus it seems probable, that the auto-reactivity against p205 induces the production of rheumatoid factors in RA.

This summary of RA-associated autoreactivities shows, that many different auto-antigens become targets of the immune system during the process of RA. These auto-antigens to different degrees also become targets of the immune system in case of other rheumatoid and non-rheumatoid diseases and even in the healthy state. Is thus has to be stated, that—according to the present knowledge—no autoreactivity by itself is suitable to improve the diagnostics of RA, neither in the early state, nor in its course or for monitoring a respective therapy.

Character of the Invention

The invention has the object to improve and support the diagnosis and therapy of chronic inflammatory joint diseases. This object is achieved by providing the “Tools for the diagnostics, molecular definition and therapy development for chronic inflammatory joint diseases” and other inflammatory, infectious or tumorous diseases. These tools are described in the following.

High-Throughput methods like DNA-array or protein array technology allow for the simultaneous detection of a large number of different parameters (9). Gene expression can be analyzed on the mRNA level by means of DNA-arrays via the hybridization of labeled RNA- or cDNA-samples, and on the protein level by arrays comprising selected protein-specific antibodies (10). Moreover, immunologic reactivities can be accessed by arrays comprising selected antigens (11).

At first, it is necessary to define the genes and proteins, which are relevant for the disease, and which are thus employed for the evaluation.

The tools according to the invention, being designed for diagnostics and therapy development for inflammatory joint diseases, are based on a such defined selection of parameters (table 1 and 2). Employing the genes given herein for a gene expression analysis by an array-method allows for a fundamentally new diagnostic approach.

For DNA-arrays intended for the determination of specific mRNA expression patterns in arthritic diseases, the genes given in table 1 can be employed in their entirety, as well as all of the genes, which are coding for proteins mentioned in table 2. Moreover, one can employ genes or partial sequences of individual genes or a selection of the genes/partial sequences given in table 1, as well as genes or partial sequences of individual genes/partial sequences or a selection of genes/partial sequences, which are coding for the proteins mentioned in table 2.

For characterizing the autoimmune reactivities, the proteins mentioned in table 2 can be used in their entirety, as well as proteins being encoded by the genes given in table 1. Moreover, also a limited selection of these proteins, selected parts of the proteins (in the form of oligo-peptides or polypeptides) or modified forms thereof may be employed. On the protein level, one also and in particular has to consider posttranslational modifications (e.g. glycosylation, phosphorylation, etc.), which can be relevant for a distinction between rheumatic diseases. The proteins, partial protein sequences and modified proteins and modified partial protein sequences are—individually, in groups or altogether—applied on a carrier matrix, which is suitable to test the patient's antibodies for their reactivity against one or several of these components. In consequence, one obtains a profile of reactivities or non-reactivities for a patient. The crucial difference between the prior art diagnostics and the diagnostic approach presented herein is the determination and analysis of one single auto-reactivity in each case in the prior art and the determination and analysis of a multitude of auto-reactivities according to the invention. The invention makes use of the unexpected finding, that combining several auto-reactivities—which are insusceptible when regarded alone—to one or more profiles, allows for a differentiation, because this approach may e.g. distinguish between a RA and a non-RA (i.e. other rheumatic diseases and non-rheumatic diseases and the healthy state) in 100% of the cases. The classification into distinct profiles is accomplished via a suitable algorithm, in an optimal form via a self-learning algorithm, which is capable to also incorporate later findings.

For the determination of protein expression patterns, array systems have been developed from protein-specific antibodies. By labelling the proteins from a protein extraction of a sample, these proteins can be quantitatively determined after having specifically bound to the corresponding antibody on the array (10). Accordingly, defined as a molecular tool in the sense of the invention is an array, which is comprised of different antibodies or molecules with a comparable protein-specific binding behaviour, being designed for the determination of all proteins or selected proteins being deduced from the genes of table 1 or for the determination of all proteins or selected proteins from table 2.

The diagnostic procedure uses biopsies from the synovial tissue, synovial fluid, blood cells, serum or plasma for the different array analyses. In this procedure, the humoral autoreactivities can be analysed in the liquid samples, the cellular autoreactivities in the blood or synovial tissue cells. The protein expression can be analysed in all of the mentioned samples, the gene expression on the mRNA level in the synovial tissue, in cells of the synovial fluid or in blood cells.

For the analysis by means of DNA-arrays, RNA is extracted from the tissue or from the cell samples derived from blood or the synovial fluid. A sample for the DNA-array hybridisation is prepared under the employment of standard protocols for amplifying (12) and labelling the derived cDNA or cRNA (13).

The genes mentioned in the table, via their known sequences (see accession number GeneBank—http://www.ncbi.nlm.nih.gov/) provide the basis, starting from which specific probes are derived for every gene. These probes are combined in an array, either by applying the prepared probes by specific printing processes (14) or by site-specific synthesis like in the photolithography on a solid phase (15, 16).

Hybridising of the labeled sample on the array provides quantitative signals via the site- and gene-specific binding, whereat these signals can be translated into an expression profile/-pattern. These patterns are correlated with established methods of evaluation, including the histological features and the classification. By an additional comparison with different joint diseases like osteoarthritis, psoriasis-associated arthritis, reactive arthritic diseases and other, partly also non-differentiated arthritic diseases, this allows for dividing the patients into different groups according to the respective expression profile.

Novelty of the Approach

In order to define trustworthy parameters for the array analysis, which allow for a classification and evaluation of the joint diseases, extensive comparative studies were performed. For this aim, different joint diseases were taken into consideration and a novel combination of different methods, partly complementing each other, was chosen.

Thus, synovial tissue from RA, osteoarthrosis and healthy joints was analysed. In order to accomplish a differential analysis of gene expression, at first the “representational difference analysis” (17, 18) was performed. This technique offers the advantage, that all mRNAs being present in the sample are encompassed, even when their sequence is yet unknown. As a drawback, it leads to an intensive selection of the most strongly pronounced differences of expression. Complementary thereto, we also tested the gene expression by means of two different methods of DNA-array-hybridisation, on the one hand on cDNA filter-arrays (19), on the other hand on oligonucleotide micro-arrays (U.S. Pat. Nos. 5,445,934; 5,744,305; 5,700,637 and 5,945,334, and furthermore EP 619321 and 373203). These micro-arrays, according to the current state of knowledge, allow to consider nearly all known human genes and to perform a comparative analysis of expression between the tissue samples for each of these individual genes. Finally, the differential gene expression for selected genes was verified in a lager sample collective by means of semiquantitative polymerase chain reaction (PCR, real-time PCR). Furthermore, tissues were characterized histologically and—according to the histological classification—also compared to the respective differential gene expression pattern. The genes given in table 1 were identified as the differentially expressed genes both between the different chronic joint diseases and in comparison to normal synovial tissue. Thus, these genes are significant for characterizing the chronic joint diseases.

Thus, there also exists a novelty in the selected approach used to identify the relevant genes. The list of the identified genes furthermore shows, that most of the genes have so far not been correlated with inflammatory rheumatoid joint diseases, and it also shows novel evaluation criteria for the diagnostics, investigation of pathophysiology and treatment of chronic joint diseases.

The characteristics of the invention are disclosed and specified by the elements of the claims and by the description, whereat both single characteristics and also several characteristics in the form of combinations constitute favorable embodiments, for which a legal protection is applied for by this specification. These characteristics are comprised of known elements—the genes or partial sequences mentioned in table 1 and the genes and partial sequences coding for the proteins mentioned in table 2—and novel elements—the novel tools being based on the employment of a defined selection of parameters (tables 1 and 2) -, which in their combination lead to the tools according to the invention, and which, under the employment of the mentioned genes for the gene expression analysis in the array method, allow for a basically new approach of diagnostics and therapy development in inflammatory joint diseases.

The tools according to the invention are based on the employment of a high-throughput method of (micro-) array hybridisation and/or a high throughput method using techniques of the polymerase chain reaction for (semi-)quantification.

They are furthermore characterized in that they are based on the use of a labeled sample derived from a patient and the use of a second, differently labeled control sample, which is used for a comparative double hybridisation to a (micro-) array together with the patient sample (comparative red/green hybridisation). The samples may also be analysed on separate arrays and compared thereafter.

According to the invention, these are tools for diagnostic purposes, which are based on the employment of
individual, a selection of, or the entirety of the proteins or peptides deduced from the gene sequences mentioned in claims 1 to 3,
individual proteins, a selection of proteins of all proteins mentioned in table 2, and
partial sequences derived from individual proteins, from a selection of proteins, or from all proteins mentioned in table 1.

They include proteins or partial protein sequences, which have sequences being identical with those of the deduced proteins of table 1 or with those of the proteins mentioned in table 2, or display a respective sequence identity of at least 80%. They are furthermore characterized in that they are based on the use of
High-throughput methods in the analytics of protein expression (high definition two-dimensional protein gel electrophoresis, MALDI techniques),
High-throughput methods in the field of the protein spotting techniques (protein arrays) designed to screen for auto-antibodies as a diagnostic tools for inflammatory joint diseases and other inflammatory, infectious or tumorous diseases in the human,
High-throughput methods in the field of the protein spotting techniques (protein arrays) designed to screen for autoreactive T cells as a diagnostic tools for inflammatory joint diseases and other inflammatory, infectious or tumorous diseases in the human, and
Non-high-throughput methods in the field of the protein spotting techniques designed to screen for autoreactive T cells as a diagnostic tools for inflammatory joint diseases and other inflammatory, infectious or tumorous diseases in the human.

The tools according to the invention are furthermore based on the employment of
antibodies, which are specific for proteins or partial sequences mentioned in claims 6 to 9, and
the respective homologous sequences of another species for the analytics in animal experiments or for the diagnostics in animals with inflammatory joint diseases and other inflammatory, infectious or tumorous diseases.

The tools according to the invention are useful as diagnostic means for the detection of genetic alterations (mutations)
in the genes or the regulatory sequences (promoter, enhancer, silencer, specific sequences for binding further regulatory factors) of the genes mentioned in claims 1 to 3, and
in the genes or the regulatory sequences (promoter, enhancer, silencer, specific sequences for binding further regulatory factors) of the genes coding for the proteins mentioned in table 2.

Moreover, these tools are suitable as means for the molecular definition of inflammatory joint diseases and other inflammatory, infectious or tumorous diseases in the human, thereby making use of the genes, DNA-sequences or the deduced corresponding proteins or peptides mentioned in claims 1 to 3, and the proteins and partial protein sequences from claims 6 to 9 or the respective coding gene sequences.

The tools according to the invention are moreover employed for
the choice of a therapy for inflammatory joint diseases and other inflammatory, infectious or tumorous diseases in the human, thereby employing the genes, DNA-sequences or deduced corresponding proteins or peptides mentioned in claims 1 to 3,
the monitoring of the progression/therapeutic success in inflammatory joint diseases and other inflammatory, infectious or tumorous diseases in the human, thereby employing the genes, DNA-sequences or deduced corresponding proteins or peptides mentioned in claims 1 to 3,
molecular means for the development of therapy concepts, which comprise a direct or indirect impact on the expression of the genes or gene sequences mentioned in claims 1 to 3,
the development of therapy concepts, which comprise a direct or indirect impact on the expression of the proteins or partial protein sequences mentioned in claims 6 to 9,
the development of therapy concepts, which comprise a direct or indirect impact on autoreactive T cells being directed against proteins or partial protein sequences mentioned in claims 8 to 11,
the impact on the biological effect of the proteins deduced from the gene sequences mentioned in claims 1 to 3.
the impact on the direct molecular regulatory pathways/circuits, in which the genes mentioned in claims 1 to 3 and the proteins deduced thereof are taking part the development of therapy concepts with the creation and employment of interpretation algorithms, thereby using the mentioned genes and sequences and their regulatory mechanisms, in order to recognize or predict therapy concepts, therapeutic effects, therapeutic optimizations or disease prognostications
the development of biologically active drugs (Biologicals) under employment of the genes, gene sequences, regulation of genes or gene sequences, or under employment of proteins, protein sequences, fusion proteins according to claims 1 to 3 and 6 to 9, or under employment of antibodies or autoreactive T cells according to claims 10 to 14.

The use of the claimed tools according to the invention is to be found in the
analysis of blood samples or tissue samples in medical diagnostics,
application in analytics according to example 1, and the
application for therapy concepts according to example 2.

Materials and Methods

Patients and Tissue Asservation

All patients were selected according to the ACR-criteria for RA (1) and OA (20). Synovial tissue was immediately transported in RPMI medium (RPMI—conventional cell culture medium, diluting medium RPMI 1640; Moore, G. E. et al., J. Am. Assoc. 199, 519-524, 1967), supplemented with penicillin and streptomycin (100 U/ml each), from the operating room into the laboratory. After the preparation of the synovial membrane, the samples were immediately shock frozen in liquid nitrogen. The samples were stored at −80° C. until further use. As samples for the Representational Difference Analysis (RDA), the hybridisation to Unigene filter arrays (http://www.ncbi.nlm.nih.gov/UniGene/) and the hybridisation to Affymetrix arrays, we used synovial tissue samples derived from normal donor (ND), osteoarthrosis (OA) and rheumatoid arthritis (RA).

Isolation of RNA

The samples were homogenized in order to extract RNA: Tissue amounts of <50 mg were crushed to powder by means of mortar and pestle while cooling with liquid nitrogen, followed by the lysis in a guanidine-isothiocyanate containing solution (RLT-buffer from Qiagen, Hilden, Germany—www.qiagen.com/literature/handbooks/rna/my96/1019545_PREHB_RNY96_prot2.pdf). Larger amounts of tissue were crushed up by means of a tissue homogenizer (IKA-Ultra-Turrax T 25; Jahnke & Kunkel, Staufen) in an icecold, guanidine-isothiocyanate containing solution (RLT-buffer from Qiagen, Hilden, Germany). The isolation of RNA was accomplished by a modified protocol using the phenol-chloroform-extraction according to Chomczynski (21), followed by the immediate isolation of RNA from the aqueous phase by means of the QIAGEN-RNaesy-Kit (see handbook of the manufacturer: http://www.qiagen.com/literature/rnalit.asp#mini). The kit was used according to the manufacturer's protocol. The RNA was eluted in 30-100 μl of RNAse-free water.

For a quality control, the optical density (OD) was measured at 260 nm (OD260), the relation of OD260/OD280 nm was determined and a gel electrophoresis was performed on 1% agarose. DNA-contaminations—if necessary—were able to be detected either in the gel or, after the first strand synthesis, in a PCR using an intron-primer for the Glycerol-aldehyde-3-phosphate dehydrogenase (GAPDH). In these exceptional cases we also digested with DNAse, thereby following the instructions of the QIAGEN protocol.

First Strand Synthesis

The cDNA-synthesis was performed under employment of the Superscript II Reverse Transcriptase (RT), including the 5× reaction buffer from Invitrogen/Life Technologies (Karlsruhe, Germany; http://www.invitrogen.com). The employed amounts of RNA were 3-5 μg for the semiquantitative PCR and 10-20 μg for the RDA and the array hybridisations in a final volume of 20 μl. The reaction mix for the transcription into cDNA contained the following components: 500 ng of each respective primer oligonucleotide (Oligo(dT)₁₂₋₁₈; T7-Oligo (dT₂₄)), 50 mM Tris pH 8,3, 75 mM KCl, 3 mM MgCl₂, 10 mM dithiothreitol, deoxynucleotide-triphosphate (dNTP) mixture with each nucleotide in a final concentration of 1 mM, 40 U RNase inhibitor and 20 U Superscript™ II RT. The incubation period was 1,5 hours, followed by the inactivation of the enzymes by heating the samples to 72° C. for 15 min.

Second Strand Synthesis

The following components were added to the cDNA by pipetting: 90 μl aqua dest., 30 μl 5× Second strand buffer (500 mM KCl, 50 mM ammonium acetate, 25 mM MgCl₂, 0,75 mM beta-nicotinamide-adenine-dinucleotide (P-NAD) and 0,25 mg/ml of bovine serum albumin (BSA)), 3 μl of a 10 mM dNTP-solution and an enzyme solution of the following activities and amounts: 1 μl E. coli ligase (10 U/μl), 4 μl DNA polymerase I (10 U/μl) and 1 μl RNAseH (2U/μl) (Invitrogen/Life Technologies, Karlsruhe, Germany). The incubation period was 2 hours at a temperature of 16° C. After having added 2 μl of a T4 DNA polymerase (5 U/μl), the incubation was pursued for further 30 min at 16° C.

Subtractive Hybridisation and RDA

The PCR Suppression Subtractive Hybridisation (SSH) (22) was performed according to the instructions of the manufacturer of the PCR Select Kit (Clontech, Palo Alto, USA; http://www.clontech.com/pcr-select/index.shtml). The digest of the double-stranded cDNA was accomplished with the restriction enzyme RsaI from Rhodopseudomonas sphaeroides. For the RDA (18), the double-stranded cDNA was cut with the restriction enzyme DPNII from Diplococcus pneumoniae (20 U in 100 μl). Then, a ligation to adapter primers (RBgl12, RBgl24) was performed, followed by amplification according to published protocols (17, 18). The tester-amplicon was obtained after a further restriction digest with DPNII by means of a ligation to a further adapter oligonucleotide (JBgl12 and JBgl24 or NBgl12 and NBgl24(18)) in the second round of subtraction.

After the hybridisation, the sequences belonging to the tester were selectively amplified by PCR and thereby accumulated in the subtraction product in both methods.

Description of the Subtraction Samples

The RDA protocols were such modified, that it became possible to identify both genes being expressed in a weaker manner and in a more pronounced manner in the samples derived from RA, OA and normal tissue donors.

In this procedure:
1 OA (driver) was subtracted from RA (tester) in order to obtain sequences, which show a stronger expression in RA- than in OA-tissues
2 RA (driver) was subtracted from ND (tester) in order to obtain sequences, which show a weaker expression in RA-samples than in ND-samples
3 ND (driver) was subtracted from OA (tester) in order to obtain sequences, which show a stronger expression in OA- than in ND-tissues

Performance of the subtraction library cloning, sequence determination and comparison to data bases

The subtraction products of the SSH-sample were cloned into a pCRII vector (TA-Cloning Kit; Invitrogen, Heidelberg, Germany; http://www.invitrogen.com). The subtraction products from the RDA were cloned into a pBluescript KS⁺II vector (Stratagene, La Jolla, USA; http://www.stratagene.com/vectors/selection/plasmid1.htm), which had previously been cut with the restriction enzyme BamHI from Bacillus amyloliquefaciens, then being dephosporylated and purified. About 150 clones were isolated and the sequence determined by means of an ABI 377 Sequencer (Applied Biosystems, Weiterstadt, Germany; http://home.appliedbiosystems.com). The sequence determination was performed according to the manufacturer's Dye Terminator Chemistry protocol under employment of a T7-primer.

After the elimination of vector sequences, the comparative analysis of the sequences was performed under employment of the Genebank and NCBI-databases (http://www.ncbi.nlm.nih.gov).

Microarray Hybridisation

Two different chip technologies were used: 1.) Use of filters, onto which the PCR-products of cDNA-clones of the UNIGENE library (http://www.ncbi.nlm.nih.gov/UniGene/) were spotted. The hybridisation was here performed at 65° C. with ³³P-labelled cDNA-samples after first strand synthesis with oligo(dT₍₁₂₋₁₈₎) (23, 24). 2.) Hybridisations were performed with the microarrays (HU95A, HU95B, HU95C, HU95D and HU95E) from Affymetrix (Affymetrix Inc., Santa Clara, USA; www.affymetrix.com). These arrays are arrays of oligonucleotides, the base sequences of which are derived from 12.000 known genes and 24.000 Expressed Sequence Tag (EST-) entries. The synthesis of the labeled samples was accomplished according to the manufacturer's technical manual (Affymetrix Inc, Santa Clara, USA).

The fluorescence-labelled sample was synthesized after transcription with an oligo-dT₂₄-primer, which contains a T7 polymerase binding site. The labelling reaction was accomplished under employment of the T7 RNA polymerase and biotinylated dNTPs according to the manufacturer's protocol (ENZO-Biochem, New York, USA; http://www.enzo.com/entrance.html).

In both chip analyses, the sample to be tested and the reference sample were hybridized to separate filters. The comparison of signal intensities was accomplished after normalization.

Evaluation of the Chip Results—Decision Matrix

The evaluation of the signal intensities was accomplished after normalization by means of the software developed for the respective array and by determining an intensity value for the respective sample according to the Tukey's Biweight Method (http://mathworld.wolfram.com/TukeysBiweight.html). For the evaluation of the Unigene filter arrays, the algorithm was developed at the Max-Planck-Institute for Molecular Genetics at Berlin-Dahlem (http://algorithms.molgen.mpg.de/). In case of the chips from Affymetrix, the MicroArraySuite 5.0 Software (httD://www.affymetrix.com/products/software/specific/mas.affx) including the manufacturer's standard parameters or preconditions was employed.

For the evaluation of the Affymetrix arrays, the target intensity was set to 100 and the normalization factor to 1 in order to normalize the data, and the scaling factor for each sample was calculated. Chips with comparable scaling factors (factor <4) were included in the comparative analyses. The decision criterion for the detection of a gene (Detection p value) was adjusted at <0,05. The comparative analyses for the respective arrays were performed under the employment of the DMT 3.0 Software from Affymatrix (htp://www.affymetrix.com/products/software/specific/dmt.affx).

Thereby, the differences between the perfect matches and the perfect- and mismatch intensities are calculated by means of the Wilcoxon-test (http://faculty.vassar.edu/lowry/wilcoxon.html) and compared to the decision criterion Cut-Off (γ-value<0,04). In the specification of results for comparison of the respective chips, a Change-Call (increased, marginally increased, no change, decreased) and the Signal Log Ratio, a measure for the factor of change, are indicated (factor in logarithmic form).

Decision Criterion

Comparative analyses were in each case performed for all samples (every sample in comparison to every sample of the other group: ND, OA, RA).

In case of the Unigene filter hybridisations, a signal difference of >2 for at least 3 of 4 comparisons, and a detection signal with a p-value <0,01 were taken into account.

The proceeding for the arrays from Affymetrix was as follows: Each RA-sample was compared to each OA-sample both in the direction of an increased and decreased expression. Genes, which in 80% of these comparisons showed a deviation in the sense of “increased” or “decreased” at a regulation factor >2 (signal log ratio >1), were selected as candidate genes. In case of the U95A chip, the selection criterion was determined to be a regulating factor >3.

Semiquantitative PCR

Starting from the detected sequence regions, we selected primers with a comparable annealing temperature and product length. For the primer search, the DNASTAR Primer Select Software (DNASTAR Inc., Madison, USA; http://www.dnastar.com/) was used. Primer synthesis was performed at Gibco-Life Technologies (Karlsruhe, Germany). For the semiquantification of the PCR-products, the real time PCR-System GeneAmp 5700 and the Sybr-Green-PCR-Core Kit (Applied Biosystems, Weiterstedt, Germany; http://europe.appliedbiosystems.com/) were employed.

The amounts of cDNA were coordinated for all samples by means of the real-time amplification results for the GAPDH-specific primers. The quantification of the PCR-products of several further genes was accomplished in relation to the GAPDH-specific product as the internal standard. As a control, β-actin as a second housekeeping gene was amplified and analysed in parallel with all samples.

| | | | Product |
|---|---|---|---|
| Gene | | | length |
| nomenclature | AccNo. | Primer-localization | (bp) |
| VDUP1 | NM006472 | 665 . . . 684/863 . . . 840 | 199 |
| TIMP4 | U76456 | 143 . . . 159/336 . . . 317 | 194 |
| GPX3 | NM002084 | 424 . . . 443/528 . . . 510 | 105 |
| β Actin | X00351- | 654 . . . 675/841 . . . 819 | 188 |
| MMP1 | X05231 | 874 . . . 895/1080 . . . 1057 | 207 |
| MMP3 | X05232 | 973 . . . 996/1157 . . . 1136 | 185 |
| LTBP4 | M22490 | 511 . . . 534/760 . . . 737 | 250 |
| GADD45 | M60974 | 457 . . . 475/573 . . . 557 | 116 |
| CLU | NM001831 | 1384 . . . 1404/1509 . . . 1489 | 126 |
| Cal2 | NM001215 | 930 . . . 949/1049 . . . 1031 | 196 |

Immunohistochemistry

A sample of the synovial membrane was used for the histopathological evaluation. Thereby, kryosections having a strength of 6 pm were prepared, air dried and then fixed with a 1:1 mixture of acetone and methanol. The hematoxylin staining was performed according to standard protocols and classified according to histopathological evaluation criteria (25).

Methods and Results of the Immunome-Analysis

Patterns of autoreactivity on the T cell and B cell level (the “immunome”) are determined, which are specific for RA and thus distinguish this disease from other rheumatic or non-rheumatic diseases. The knowledge about the RA-specific immunome is of crucial importance for the development of diagnostic tools, which recognize an arthritic disease much earlier and safer as an RA or show the arthritis not to be an RA, than it is possible nowadays. This again allows to control the RA by suitable drugs before irreversible joint and bone damages have occurred.

For this aim, techniques of Proteomics are employed in order to create tissue specific protein patterns by means of high definition 2D-electrophoresis. These were screened by techniques of Immunomics for known and unknown autoreactivities. Protein spots with a useful sensitivity and specificity are identified by sequencing and MALDI-TOF (26). These proteins are then screened for T cell autoreactivity in the same cohort.

According to the invention, autoreactivity patterns have been established, which are completely specific for RA. In this analysis, it is of a great importance, that no single autoreactivity reveals this specificity. This is only reached by the combination of several autoreactivities. Such patterns, which undoubtedly distinguish a patient with RA from a patient suffering from another rheumatic or non-rheumatic disease, comprise the auto-antigens citrullinated peptides (Cit), IgG, BiP (Heavy Chain Binding Protein), Calpastatin (Calp), RA33 (hnRNP A2) and Calreticulin (Calr). The table shows all possible combinations of five of these autoreactivities (RF, Cit, BiP, RA33 and Calp) and the two possible conditions “positive” and “negative”. The highlighted patterns (statistically relevant, p<0,01, Whitney U Test; http://faculty.vassar.edu/lowry/utest.html) are only expressed in RA. FIG. 1 shows the sensitivities for all possible combinations both for RA and the control cohorts. The RA-specific patterns are highlighted in a manner analogous to table 1 and mainly comprise those, which are fourfold and fivefold positive for the individual parameters. The combination of those autoreactivity profiles, which only occur in RA, yields a specificity of 54%.

Exclusively RA-expressed patterns of the three autoreactivities, which are directed against IgG, Cit and BiP (RF+Cit+BiP+ and RF−Cit+BiP+) yield a total sensitivity of 43%. RA-exclusive patterns of the four autoreactivities, which are directed against IgG, Cit, BiP and RA33 (RF+Cit+Bip+RA33+, RF+Cit+BiP−RA33+ and RF+Cit+BiP+RA33−) show a total sensitivity of 40%. In the analysis of six patterns, a sensitivity of 60% is achieved.

According to first investigations, these patterns are also relevant for patients with early RA. Further candidate antigens, which have already been characterized, comprise the Sa-antigen (5), which probably consists of α-Enolase and citrullinated Vimentin.

The identification of the immunome of RA not only is of diagnostic, but also of pathogenetic relevance. When those T cellular autoreactivities being responsible for driving the early RA are identified, it appears to be possible to develop protocols for therapy, which display a specific effectiveness.

Scheme 1: Patterns of autoreactivity with RF, Citrullin, BiP, Calpastatin
and RA33.

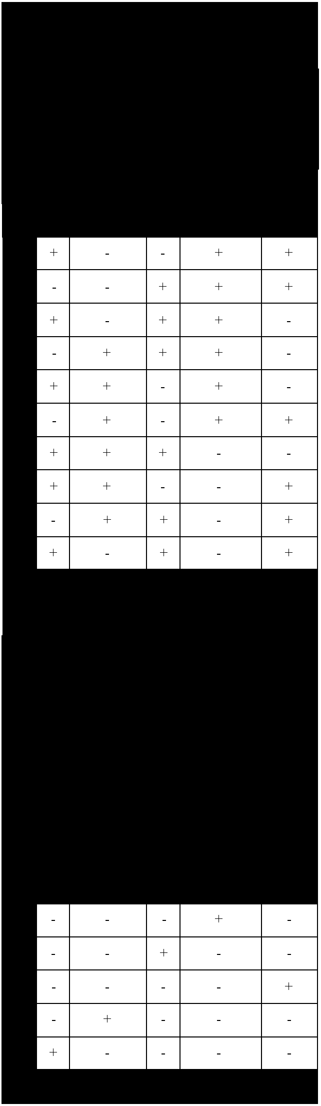

Indicated are all 32 possible quintuple combinations of the autoreactivities directed against IgG (RF), Citrullin, BiP, Calpastatin and RA33. Like in FIG. 1, the RA-specific combinations are highlighted in color.

Advantage

Complex molecular patterns are covered. These patterns can be classified by means of mathematical calculation models into groups and congeniality scales. The respective, derivable classification and knowledge about the association e.g. with the duration of the disease, the clinical disease activity (Disease Activity Score (Ref.)), the inflammatory activity being determined by the increase of the C-reactive Protein or by the sedimentation rate, the radiological joint destruction and the specific influence of drugs, allow to draw the following conclusions from the array-analysis: assignment of the clinical picture to a defined diagnosis and to a subgroup allowing to be molecularly classified, evaluation of the disease activity and the progredience to be expected prognostic evaluation), perspectives of different therapy forms, recommendation for suitable therapeutic approaches (e.g. Methotrexate instead of Leflunomide, or a combination of Sulfasalazine and Methotrexate instead of Methotrexate alone) and, finally, mon itoring of the therapeutic success.

By the employment before and during de fined measures of medicinal treatment , it can be determined, which of the employed ge nes are affected by the drug. It is t hereby measured, how the dru g affects the gene expre ssion being altered in a disease-typical ma nner. Starting from this, it can be concluded, which disease-related molecular alterations are still valid in defiance of the therapy. The knowledge about the function of these pathologically active genes principally allows to elucidate pathophysiological processes of the joint disease and to deduce novel therapy concepts.

Combination of the Genes

TABLE 1

| Accession | Unigene | | | |
|---|---|---|---|---|
| number | codification | Name of the Gene | Method | Regulation |
| X57809 | Hs.181125 | | RDA, | RA > OA |
| | | | Affymetrix | |
| X58141 | | | RDA | RA > OA |
| X63527 | Hs.252723 | ribosomal protein L19 | RDA | RA > OA |
| U10362 | Hs.75864 | chromosome 5 open | RDA | RA > OA |
| | | reading frame 8 | | |
| M80244 | Hs.184601 | NM_003486 | RDA | RA > OA |
| M24594 | Hs.20315 | interferon-induced protein | RDA | OA > RA |
| | | with tetratricopeptide | | |
| | | repeats 1 | | |
| U01244 | Hs.79732 | fibulin 1 isoform C | RDA | RA > OA |
| | | precursor NM_006485 | | |
| X02761 | Hs.287820 | fibronectin 1, isoform 1 | RDA | RA > OA, |
| | | preproprotein | | OA > NS |
| L01124 | Hs.165590 | ribosomal protein S13 | RDA | NS > RA |
| M65062 | Hs.107169 | insulin-like growth factor | RDA | |
| | | binding protein 5 | | |
| M15330 | Hs.126256 | interleukin 1, beta | | |
| L13210 | Hs.79339 | galectin 3 binding protein | RDA | |
| X05232 | Hs.83326 | matrix metalloproteinase 3 preproprotein | | RA > NS, OA |
| M22490 | Hs.68879 | bone morphogenetic | RDA, | NS > RA |
| | | protein 4 | Affymetrix | |
| AL034397 | | | RDA | OA > NS |
| M22806 | | | RDA | OA > NS |
| X06256 | Hs.149609 | integrin alpha 5 precursor | Unigene | NS > RA |
| L49169 | Hs.75678 | FBJ murine osteosarcoma | Unigene | NS > RA |
| | | viral oncogene homolog B | | |
| AB002409 | Hs.57907 | small inducible cytokine | Unigene | RA > NS |
| | | subfamily A (Cys—Cys), | | |
| | | member 21 | | |
| X03473 | Hs.226117 | H1 histone family, | RDA | OA > NS |
| | | member 0 | | |
| M92843 | Hs.343586 | zinc finger protein 36, | Unigene | NS > RA |
| | | C3H type, homolog | | |
| | | (mouse) | | |
| M21121 | Hs.241392 | small inducible cytokine | Affymetrix | RA > OA |
| | | A5 (RANTES) | | |
| U05259 | | | Affymetrix | RA > OA |
| U80114 | Hs.247987 | | Affymetrix | RA > OA |
| U81234 | Hs.164021 | small inducible cytokine | Affymetrix | RA > OA |
| | | subfamily B (Cys-X-Cys), | | |
| | | member 6 (granulocyte | | |
| | | chemotac | | |
| D11086 | Hs.84 | interleukin 2 receptor, | Affymetrix | RA > OA |
| | | gamma chain, precursor | | |
| X97267 | | | Affymetrix | RA > OA |
| U23852 | | | Affymetrix | RA > OA |
| AA522530 | Hs.111244 | RTP801 | Affymetrix | RA > OA |
| AF037335 | Hs.5338 | carbonic anhydrase XII | Affymetrix | RA > OA |
| | | precursor | | |
| U97145 | Hs.19317 | GDNF family receptor | Affymetrix | RA > OA |
| | | alpha 2 | | |
| AA919102 | Hs.95327 | CD3D antigen, delta | Affymetrix | RA > OA |
| | | polypeptide (TiT3 | | |
| | | complex) | | |
| M63928 | Hs.180841 | CD27 antigen | Affymetrix | RA > OA |
| Z49194 | Hs.2407 | POU domain, class 2, | Affymetrix | RA > OA |
| | | associating factor 1 | | |
| AL031983 | | | Affymetrix | RA > OA |
| D15050 | Hs.232068 | | Affymetrix | RA > OA |
| X92997 | Hs.342651 | | Affymetrix | RA > OA |
| J03910 | | | Affymetrix | RA > OA |
| J04132 | Hs.97087 | T-cell receptor zeta chain | Affymetrix | RA > OA |
| | | precursor | | |
| M55153 | Hs.8265 | transglutaminase 2 (C | Affymetrix | RA > OA |
| | | polypeptide, protein- | | |
| | | glutamine-gamma- | | |
| | | glutamyltransferase) | | |
| M12959 | Hs.74647 | | Affymetrix | RA > OA |
| AF031815 | Hs.89230 | potassium intermediate | Affymetrix | RA > OA |
| L31584 | | | Affymetrix | RA > OA |
| X54489 | | | Affymetrix | RA > OA |
| AF043129 | | | Affymetrix | RA > OA |
| X59871 | Hs.169294 | transcription factor 7 (T- | Affymetrix | RA > OA |
| | | cell specific, HMG-box) | | |
| AI743134 | Hs.21858 | trinucleotide repeat | Affymetrix | RA > OA |
| | | containing 3 | | |
| Y13323 | Hs.145296 | disintegrin protease | Affymetrix | RA > OA |
| U77735 | Hs.80205 | pim-2 oncogene | Affymetrix | RA, OA > NS |
| U58515 | Hs.154138 | chitinase 3-like 2 | Affymetrix | RA, OA > NS |
| M17016 | Hs.1051 | granzyme B precursor | Affymetrix | RA > OA |
| X03066 | Hs.1802 | major histocompatibility | Affymetrix | RA, OA > NS |
| | | complex, class II, DO beta | | |
| M28170 | Hs.96023 | CD19 antigen | Affymetrix | RA, OA > NS |
| L24564 | Hs.1027 | Ras-related associated | Affymetrix | NS > RA |
| | | with diabetes | | |
| M68840 | Hs.183109 | monoamine oxidase A | Affymetrix | NS > RA |
| U76456 | Hs.190787 | tissue inhibitor of | Affymetrix | OA > RA |
| | | metalloproteinase 4 | | |
| | | precursor | | |
| D13814 | Hs.89472 | angiotensin receptor 1 | Affymetrix | NS > RA |
| | | NM_004835 | | |
| AA420624 | Hs.183109 | monoamine oxidase A | Affymetrix | OA > RA |
| X51757 | Hs.3268 | heat shock 70 kD protein 6 | Affymetrix | NS > RA |
| | | (HSP70B′) | | |
| U29344 | Hs.83190 | fatty acid synthase | Affymetrix | NS > RA |
| L19871 | Hs.460 | activating transcription | Affymetrix | NS > RA |
| | | factor 3 long isoform | | |
| | | NM_004024 | | |
| J02611 | Hs.75736 | apolipoprotein D | Affymetrix | NS > RA |
| | | precursor | | |
| M12272 | Hs.2523 | class I alcohol | Affymetrix | NS > RA |
| | | dehydrogenase, gamma | | |
| | | subunit | | |
| L34041 | Hs.348601 | glycerol-3-phosphate | Affymetrix | NS > RA |
| | | dehydrogenase 1 (soluble) | | |
| L12760 | Hs.1872 | phosphoenolpyruvate | Affymetrix | OA > RA |
| | | carboxykinase 1 (soluble) | | |
| M63978 | | | Affymetrix | RA > OA |
| S95936 | Hs.284176 | transferrin precursor | Affymetrix | NS > RA |
| U42031 | Hs.7557 | FK506-binding protein 5 | Affymetrix | NS > RA |
| Z97171 | | | Affymetrix | NS > RA |
| S69790 | | | Affymetrix | NS > RA |
| U41843 | Hs.295362 | DR1-associated protein 1 | Affymetrix | OA, NS > RA |
| | | (negative cofactor 2 | | |
| | | alpha) | | |
| AL049653 | | | Affymetrix | NS > RA |
| M31682 | Hs.1735 | inhibin beta B subunit | Affymetrix | NS > RA |
| | | precursor | | |
| AF009767 | Hs.132898 | fatty acid desaturase 1 | Affymetrix | NS > RA, OA |
| X02910 | Hs.241570 | tumor necrosis factor (cachectin) | | |
| AB023152 | Hs.12183 | | Affymetrix | NS > RA, OA |
| U37283 | Hs.300946 | Microfibril-associated | Affymetrix | OA, NS > RA |
| | | glycoprotein-2 | | |
| X05451 | Hs.158295 | | Affymetrix | OA, NS > RA |
| W26480 | Hs.132898 | fatty acid desaturase 1 | Affymetrix | NS > RA |
| D14874 | Hs.394 | adrenomedullin | Affymetrix | RA > NS |
| M12174 | Hs.204354 | ras homolog gene family, | Affymetrix | NS > RA |
| | | member B | | |
| M60974 | Hs.80409 | growth arrest and DNA- | Affymetrix | NS > RA |
| | | damage-inducible, alpha | | |
| S62138 | | | Affymetrix | NS > RA |
| X16706 | Hs.301612 | FOS-like antigen 2 | Affymetrix | NS > RA |
| X56667 | Hs.106857 | calbindin 2, full length | Affymetrix | NS > RA |
| | | protein isoform | | |
| | | NM_007087 | | |
| H15814 | | | Affymetrix | NS > RA |
| AL021977 | | | Affymetrix | NS > RA |
| U80055 | | | Affymetrix | NS > RA |
| U09564 | Hs.75761 | SFRS protein kinase 1 | Affymetrix | RA > OA |
| U14407 | Hs.168132 | interleukin 15 | Affymetrix | RA > OA |
| U27185 | Hs.82547 | retinoic acid receptor | Affymetrix | RA > OA |
| | | responder (tazarotene | | |
| | | induced) 1 | | |
| Z35278 | Hs.170019 | runt-related transcription | Affymetrix | RA > OA |
| | | factor 3 | | |
| M12886 | Hs.303157 | | Affymetrix | RA > OA |
| L05424 | | | Affymetrix | RA > OA |
| L09230 | Hs.301921 | chemokine (C—C motif) | Affymetrix | RA > OA |
| | | receptor 1 | | |
| L22075 | Hs.1666 | guanine nucleotide | Affymetrix | RA > OA |
| | | binding protein (G | | |
| | | protein), alpha 13 | | |
| M28130 | | | Affymetrix | RA > OA |
| M29696 | Hs.237868 | interleukin 7 receptor | Affymetrix | RA > OA |
| M31165 | Hs.29352 | tumor necrosis factor, | Affymetrix | RA > OA |
| | | alpha-induced protein 6 | | |
| M16038 | Hs.80887 | v-yes-1 Yamaguchi | Affymetrix | RA > OA |
| | | sarcoma viral related | | |
| | | oncogene homolog | | |
| X83490 | | | Affymetrix | RA > OA |
| D13666 | Hs.136348 | osteoblast specific factor 2 | Affymetrix | RA > OA |
| | | (fasciclin I-like) | | |
| L10717 | Hs.211576 | IL2-inducible T-cell | Affymetrix | RA > OA |
| | | kinase | | |
| X04500 | Hs.126256 | interleukin 1, beta | Affymetrix | RA > OA |
| U24153 | Hs.30692 | p21 (CDKN1A)-activated | Affymetrix | RA > OA |
| | | kinase 2 | | |
| M32315 | Hs.256278 | tumor necrosis factor | Affymetrix | RA > OA |
| | | receptor 2 (75 kD) | | |
| U51903 | Hs.78993 | IQ motif containing | Affymetrix | RA > OA |
| | | GTPase activating protein 2 | | |
| AF002700 | Hs.19317 | GDNF family receptor | Affymetrix | RA > OA |
| | | alpha 2 | | |
| U37518 | Hs.83429 | tumor necrosis factor | Affymetrix | RA > OA |
| | | (ligand) superfamily, | | |
| | | member 10 | | |
| HG1103-HT1103 | | | Affymetrix | RA > OA |
| HG3521-HT3715 | | | Affymetrix | RA > OA |
| AF024710 | | | Affymetrix | RA > OA |
| U01134 | Hs.138671 | fms-related tyrosine | Affymetrix | RA > OA |
| | | kinase 1 (vascular | | |
| | | endothelial growth factor | | |
| U27467 | Hs.227817 | BCL2-related protein A1 | Affymetrix | RA > OA |
| M79321 | Hs.80887 | v-yes-1 Yamaguchi | Affymetrix | RA > OA |
| | | sarcoma viral related | | |
| | | oncogene homolog | | |
| J04765 | Hs.313 | secreted phosphoprotein 1 | Affymetrix | RA > OA |
| | | (osteopontin, bone | | |
| | | sialoprotein I, early T- | | |
| | | lymphocyte | | |
| M21154 | Hs.262476 | S-adenosylmethionine | Affymetrix | RA > OA |
| | | decarboxylase 1 precursor | | |
| AF098641 | Hs.306278 | | Affymetrix | RA > OA |
| D63789 | Hs.174228 | small inducible cytokine | Affymetrix | RA > OA |
| | | subfamily C, member 2 | | |
| S68134 | Hs.351252 | cAMP responsive element | Affymetrix | RA > OA |
| | | modulator | | |
| AB014515 | Hs.323712 | KIAA0615 gene product | Affymetrix | RA > OA |
| AI800499 | Hs.161002 | | Affymetrix | RA > OA |
| Y13710 | Hs.16530 | small inducible cytokine | Affymetrix | RA > OA |
| | | subfamily A (Cys—Cys), | | |
| | | member 18, pulmonary | | |
| | | and activat | | |
| AJ011915 | Hs.184376 | synaptosomal-associated | Affymetrix | RA > OA |
| | | protein, 23 kD | | |
| AF030339 | Hs.286229 | plexin C1 | Affymetrix | RA > OA |
| X17042 | Hs.1908 | proteoglycan 1, secretory | Affymetrix | RA > OA |
| | | granule | | |
| AF059214 | Hs.194687 | cholesterol 25- | Affymetrix | RA > OA |
| | | hydroxylase | | |
| D42043 | Hs.79123 | | Affymetrix | RA > OA |
| M24283 | Hs.168383 | intercellular adhesion | Affymetrix | RA > OA |
| | | molecule 1 precursor | | |
| AF042729 | Hs.171776 | inositol(myo)-1(or 4)- | Affymetrix | RA > OA |
| | | monophosphatase 1 | | |
| M64595 | Hs.173466 | ras-related C3 botulinum | Affymetrix | RA > OA |
| | | toxin substrate 2 | | |
| AA868382 | Hs.198253 | major histocompatibility | Affymetrix | RA > OA |
| | | complex, class II, DQ | | |
| | | alpha 1 | | |
| AB006746 | Hs.198282 | phospholipid scramblase 1 | Affymetrix | RA > OA |
| X00437 | Hs.303157 | | Affymetrix | RA > OA |
| M59287 | | | Affymetrix | RA > OA |
| AA725102 | Hs.51305 | v-maf | Affymetrix | RA > OA |
| | | musculoaponeurotic | | |
| | | fibrosarcoma oncogene | | |
| | | homolog F (avian) | | |
| M97935 | Hs.21486 | signal transducer and | Affymetrix | RA > OA |
| | | activator of transcription | | |
| | | 1, 91 kD | | |
| X54134 | Hs.31137 | protein tyrosine | Affymetrix | RA > OA |
| | | phosphatase, receptor | | |
| | | type, E | | |
| U89942 | Hs.83354 | lysyl oxidase-like 2 | Affymetrix | RA > OA |
| AF099935 | Hs.17839 | TNF-induced protein | Affymetrix | RA > OA |
| M93056 | | | Affymetrix | RA > OA |
| M97936 | | | Affymetrix | RA > OA |
| AI887421 | Hs.82547 | retinoic acid receptor | Affymetrix | RA > OA |
| | | responder (tazarotene | | |
| | | induced) 1 | | |
| D50532 | Hs.54403 | macrophage lectin 2 | Affymetrix | RA > OA |
| | | (calcium dependent) | | |
| AI813532 | Hs.256278 | tumor necrosis factor | Affymetrix | RA > OA |
| | | receptor 2 (75 kD) | | |
| U02020 | Hs.239138 | pre-B-cell colony- | Affymetrix | RA > OA |
| | | enhancing factor | | |
| X05276 | Hs.250641 | tropomyosin 4 | Affymetrix | RA > OA |
| AF006516 | Hs.24752 | spectrin SH3 domain | Affymetrix | RA > OA |
| | | binding protein 1 | | |
| AB018301 | Hs.22039 | | Affymetrix | RA > OA |
| AB010812 | Hs.22900 | nuclear factor (erythroid- | Affymetrix | RA > OA |
| | | derived 2)-like 3 | | |
| AF052124 | Hs.313 | secreted phosphoprotein 1 | Affymetrix | RA > OA |
| | | (osteopontin, bone | | |
| | | sialoprotein I, early T- | | |
| | | lymphocyte | | |
| AB008775 | Hs.104624 | aquaporin 9 | Affymetrix | RA > OA |
| AF024714 | Hs.105115 | absent in melanoma 2 | Affymetrix | RA > OA |
| M28696 | Hs.278443 | Fc fragment of IgG, low | Affymetrix | RA > OA |
| | | affinity IIb, receptor for | | |
| | | (CD32) | | |
| X62573 | | | Affymetrix | RA > OA |
| X07834 | Hs.318885 | superoxide dismutase 2, | Affymetrix | RA > OA |
| | | mitochondrial | | |
| AL050267 | Hs.23889 | DKFZP564A032 protein | Affymetrix | RA > OA |
| U83461 | Hs.24030 | solute carrier family 31 | Affymetrix | RA > OA |
| | | (copper transporters), | | |
| | | member 2 | | |
| AB018285 | Hs.321707 | | Affymetrix | RA > OA |
| AF007875 | Hs.5085 | dolichyl-phosphate | Affymetrix | RA > OA |
| | | mannosyltransferase | | |
| | | polypeptide 1 | | |
| X78686 | Hs.89714 | small inducible cytokine | Affymetrix | RA > OA |
| | | subfamily B (Cys-X-Cys), | | |
| | | member 5 (epithelial- | | |
| | | derived n | | |
| AF053712 | Hs.115770 | | Affymetrix | RA > OA |
| AF006083 | Hs.5321 | ARP3 actin-related | Affymetrix | RA > OA |
| | | protein 3 homolog | | |
| AL050025 | Hs.5344 | adaptor-related protein | Affymetrix | RA > OA |
| | | complex 1, gamma 1 | | |
| | | subunit | | |
| M17017 | Hs.624 | interleukin 8 | Affymetrix | RA > OA |
| AI651024 | Hs.15780 | | Affymetrix | RA > OA |
| AF038172 | | | Affymetrix | RA > OA |
| M55542 | Hs.62661 | guanylate binding protein | Affymetrix | RA > OA |
| | | 1, interferon-inducible, | | |
| | | 67 kD | | |
| U11276 | Hs.169824 | killer cell lectin-like | Affymetrix | RA > OA |
| | | receptor subfamily B, | | |
| | | member 1 | | |
| Z19585 | Hs.75774 | thrombospondin 4 | Affymetrix | OA > RA |
| L27560 | | | Affymetrix | OA > RA |
| M98539 | | | Affymetrix | OA > RA |
| J00153 | | | Affymetrix | OA > RA |
| M25079 | Hs.155376 | hemoglobin, beta | Affymetrix | OA > RA |
| M80482 | Hs.170414 | paired basic amino acid | Affymetrix | OA > RA |
| | | cleaving system 4 | | |
| L48215 | Hs.155376 | hemoglobin, beta | Affymetrix | OA > RA |
| AA524547 | Hs.160318 | phospholemman, isoform | Affymetrix | OA > RA |
| | | b precursor NM_005031 | | |
| AL038340 | | | Affymetrix | OA > RA |
| AI381790 | Hs.74120 | adipose specific 2 | Affymetrix | OA > RA |
| X00129 | Hs.76461 | retinol-binding protein 4, | Affymetrix | OA > RA |
| | | plasma precursor | | |
| U66619 | Hs.71622 | SWI | Affymetrix | OA > RA |
| M30038 | Hs.334455 | alpha tryptase I precursor | Affymetrix | OA > RA |
| U13666 | Hs.184907 | G protein-coupled | Affymetrix | OA > RA |
| | | receptor 1 | | |
| L05144 | Hs.1872 | phosphoenolpyruvate | Affymetrix | OA > RA |
| | | carboxykinase 1 (soluble) | | |
| U39447 | Hs.198241 | copper containing amine | Affymetrix | OA > RA |
| | | oxidase 3 precursor | | |
| AL049313 | | | Affymetrix | OA > RA |
| AL050125 | | | Affymetrix | OA > RA |
| D12485 | | | Affymetrix | OA > RA |
| X78416 | Hs.3155 | casein, alpha | Affymetrix | OA > RA |
| AB028998 | Hs.6147 | | Affymetrix | OA > RA |
| AB020629 | Hs.38095 | ATP-binding cassette, | Affymetrix | OA > RA |
| | | sub-family A member 8 | | |
| X03350 | Hs.4 | class I alcohol | Affymetrix | OA > RA |
| | | dehydrogenase, beta | | |
| | | subunit | | |
| AJ224677 | Hs.7122 | scrapie responsive protein 1 | Affymetrix | OA > RA |
| AB018317 | Hs.22201 | | Affymetrix | OA > RA |
| AF009314 | | | Affymetrix | OA > RA |
| L77730 | | | Affymetrix | OA > RA |
| D76435 | Hs.41154 | Zic family member 1 | Affymetrix | OA > RA |
| | | (odd-paired homolog, | | |
| | | Drosophila) | | |
| W28828 | Hs.133988 | | Affymetrix | OA > RA |
| M73720 | | | Affymetrix | OA > RA |
| M55150 | Hs.73875 | fumarylacetoacetase | Affymetrix | OA > RA |
| U13616 | Hs.75893 | ankyrin 3, isoform 2 | Affymetrix | OA > RA |
| | | NM_020987 | | |
| AB005293 | Hs.103253 | perilipin | Affymetrix | OA > RA |
| L07765 | Hs.76688 | carboxylesterase 1 | Affymetrix | OA > RA |
| | | (monocyte | | |
| X82209 | Hs.268515 | meningioma 1 | Affymetrix | OA > RA |
| J03507 | Hs.78065 | complement component 7 | Affymetrix | OA > RA |
| | | precursor | | |
| AF013570 | Hs.78344 | smooth muscle myosin | Affymetrix | OA > RA |
| | | heavy chain 11, isoform | | |
| | | SM1 NM_022870 | | |
| U70370 | Hs.84136 | paired-like homeodomain | Affymetrix | OA > RA |
| | | transcription factor 1 | | |
| U75744 | Hs.88646 | deoxyribonuclease I-like 3 | Affymetrix | OA > RA |
| M60278 | Hs.799 | diphtheria toxin receptor | Affymetrix | OA > RA |
| | | (heparin-binding | | |
| | | epidermal growth factor- | | |
| | | like growth f | | |
| AF042166 | Hs.81008 | filamin B, beta (actin | Affymetrix | OA > RA |
| | | binding protein 278) | | |
| J00123 | | | Affymetrix | OA > RA |
| AI207842 | Hs.8272 | prostaglandin D2 synthase | Affymetrix | OA > RA |
| | | (21 kD, brain) | | |
| AA128249 | Hs.83213 | fatty acid binding protein | Affymetrix | OA > RA |
| | | 4, adipocyte | | |
| AA152406 | Hs.114346 | cytochrome c oxidase | Affymetrix | OA > RA |
| | | subunit VIIa polypeptide | | |
| | | 1 (muscle) precursor | | |
| AF093118 | Hs.11494 | fibulin 5 | Affymetrix | OA > RA |
| L38486 | Hs.296049 | | Affymetrix | OA > RA |
| U66689 | | | Affymetrix | OA > RA |
| AF049884 | Hs.350266 | Arg | Affymetrix | OA > RA |
| AB011089 | Hs.12372 | tripartite motif protein | Affymetrix | OA > RA |
| | | TRIM2 | | |
| AF060568 | | | Affymetrix | OA > RA |
| AF059293 | Hs.114948 | cytokine receptor-like | Affymetrix | OA > RA |
| | | factor 1 | | |
| AC003107 | Hs.1584 | cartilage oligomeric | Affymetrix | OA > RA |
| | | matrix protein presursor | | |
| J05037 | Hs.76751 | serine dehydratase | Affymetrix | OA > RA |
| D45371 | Hs.80485 | adipose most abundant | Affymetrix | OA > RA |
| | | gene transcript 1 | | |
| U78190 | | | Affymetrix | OA > RA |
| U24578 | Hs.444 | serine | Affymetrix | OA > RA |
| M15856 | Hs.180878 | lipoprotein lipase | Affymetrix | OA > RA |
| | | precursor | | |
| AF055033 | Hs.107169 | insulin-like growth factor | Affymetrix | OA > RA |
| | | binding protein 5 | | |
| AA976838 | Hs.268571 | apolipoprotein C-I | Affymetrix | OA > RA |
| | | precursor | | |
| L13698 | Hs.65029 | growth arrest-specific 1 | Affymetrix | OA > RA |
| AB020316 | Hs.134015 | uronyl-2-sulfotransferase | Affymetrix | OA > RA |
| U32324 | Hs.64310 | interleukin 11 receptor, | Affymetrix | OA > RA |
| | | alpha | | |
| S67070 | Hs.78846 | heat shock 27 kD protein 2 | Affymetrix | OA > RA |
| M12529 | Hs.169401 | apolipoprotein E | Affymetrix | OA > RA |
| D50495 | Hs.80598 | transcription elongation | Affymetrix | OA > RA |
| | | factor A (SII), 2 | | |
| D00632 | Hs.336920 | plasma glutathione | Affymetrix | OA > RA |
| | | peroxidase 3 precursor | | |
| AI760613 | Hs.29283 | | Affymetrix | RA > OA |
| AW014646 | Hs.303157 | | Affymetrix | RA > OA |
| W74027 | Hs.132906 | 19A24 protein | Affymetrix | RA > OA |
| W72338 | Hs.23703 | | Affymetrix | RA > OA |
| AI805006 | Hs.8882 | | Affymetrix | RA > OA |
| W67655 | | | Affymetrix | RA > OA |
| AA631460 | Hs.285814 | | Affymetrix | RA > OA |
| AI741321 | Hs.10760 | asporin (LRR class 1) | Affymetrix | RA > OA |
| AI983115 | Hs.132781 | class I cytokine receptor | Affymetrix | RA > OA |
| AI535730 | Hs.262958 | | Affymetrix | RA > OA |
| AA977937 | Hs.102308 | potassium inwardly- | Affymetrix | RA > OA |
| | | rectifying channel, | | |
| | | subfamily J, member 8 | | |
| AA447232 | Hs.334838 | | Affymetrix | RA > OA |
| AI720806 | Hs.49943 | | Affymetrix | RA > OA |
| W23237 | Hs.296162 | | Affymetrix | RA > OA |
| AI762695 | Hs.146381 | RNA binding motif | Affymetrix | RA > OA |
| | | protein, X chromosome | | |
| AI653211 | Hs.96657 | | Affymetrix | RA > OA |
| AA633405 | Hs.1101 | POU domain, class 2, | Affymetrix | RA > OA |
| | | transcription factor 2 | | |
| N78018 | Hs.267566 | hypothetical protein | Affymetrix | RA > OA |
| | | FLJ20371 | | |
| AI625959 | Hs.112242 | | Affymetrix | RA > OA |
| T66196 | Hs.111554 | ADP-ribosylation factor- | Affymetrix | RA > OA |
| | | like 7 | | |
| AI697841 | Hs.20450 | BCM-like membrane | Affymetrix | RA > OA |
| | | protein precursor | | |
| | | NM_014036 | | |
| AA569128 | Hs.283021 | chloride intracellular | Affymetrix | OA > RA |
| | | channel 5 | | |
| R53594 | Hs.260164 | | Affymetrix | OA > RA |
| AI970898 | Hs.234898 | | Affymetrix | OA > RA |
| AI972390 | Hs.348493 | | Affymetrix | OA > RA |
| N23769 | Hs.26691 | | Affymetrix | OA > RA |
| AI806324 | Hs.28625 | | Affymetrix | OA > RA |
| N28741 | Hs.75354 | | Affymetrix | OA > RA |
| AL040912 | Hs.31595 | oligodendrocyte | Affymetrix | OA > RA |
| | | transmembrane protein | | |
| AI681917 | Hs.3321 | | Affymetrix | OA > RA |
| AW006235 | Hs.41502 | hypothetical protein | Affymetrix | OA > RA |
| | | FLJ21276 | | |
| W73819 | Hs.352100 | | Affymetrix | OA > RA |
| T77033 | Hs.182364 | | Affymetrix | OA > RA |
| AW015787 | Hs.237731 | | Affymetrix | OA > RA |
| N30858 | Hs.44234 | triggering receptor | Affymetrix | OA > RA |
| | | expressed on myeloid | | |
| | | cells 2 | | |
| AI810669 | Hs.44829 | | Affymetrix | OA > RA |
| N49922 | Hs.1787 | proteolipid protein1 | Affymetrix | OA > RA |
| | | (Pelizaeus-Merzbacher | | |
| | | disease, spastic paraplegia | | |
| | | 2, uncomp | | |
| AA082546 | Hs.48516 | | Affymetrix | OA > RA |
| AI694320 | Hs.6295 | | Affymetrix | OA > RA |
| AI632283 | Hs.47448 | | Affymetrix | OA > RA |
| AA039324 | Hs.201925 | | Affymetrix | OA > RA |
| AA877186 | Hs.90250 | | Affymetrix | OA > RA |
| R42166 | Hs.94000 | | Affymetrix | OA > RA |
| AI631882 | Hs.6510 | thyrotropin-releasing | Affymetrix | OA > RA |
| | | hormone degrading | | |
| | | ectoenzyme | | |
| W68636 | Hs.168640 | ankylosis, progressive | Affymetrix | OA > RA |
| | | homolog NM_054027 | | |
| | | ankylosis, progressive | | |
| | | homolog | | |
| AA700227 | Hs.10119 | | Affymetrix | OA > RA |
| AI948584 | Hs.350495 | | Affymetrix | OA > RA |
| AI678080 | Hs.141693 | | Affymetrix | OA > RA |
| AI732274 | Hs.11006 | | Affymetrix | OA > RA |
| AI341383 | Hs.349764 | | Affymetrix | OA > RA |
| Z99386 | Hs.173638 | | Affymetrix | OA > RA |
| W95023 | Hs.173933 | | Affymetrix | OA > RA |
| AI860775 | Hs.98506 | | Affymetrix | OA > RA |
| AA464846 | Hs.103262 | | Affymetrix | OA > RA |
| AI751698 | Hs.184907 | G protein-coupled | Affymetrix | OA > RA |
| | | receptor 1 | | |
| AA545730 | Hs.293821 | | Affymetrix | OA > RA |
| AA181060 | Hs.349283 | | Affymetrix | OA > RA |
| AA195184 | | | Affymetrix | OA > RA |
| AI680541 | Hs.23767 | hypothetical protein | Affymetrix | OA > RA |
| | | FLJ12666 | | |
| AI659533 | Hs.348490 | | Affymetrix | OA > RA |
| AI750575 | Hs.173933 | | Affymetrix | OA > RA |
| AI870335 | Hs.32450 | | Affymetrix | OA > RA |
| AA160945 | Hs.14479 | | Affymetrix | OA > RA |
| AI936699 | Hs.193784 | | Affymetrix | OA > RA |
| AI130027 | Hs.293539 | | Affymetrix | OA > RA |
| AA081093 | Hs.68055 | | Affymetrix | OA > RA |
| AA142913 | Hs.71721 | | Affymetrix | OA > RA |
| AI984000 | Hs.37482 | COPZ2 for nonclathrin | Affymetrix | OA > RA |
| | | coat protein zeta-COP | | |
| AI864898 | Hs.43125 | | Affymetrix | OA > RA |
| AI670876 | Hs.44276 | homeo box C10 | Affymetrix | OA > RA |
| AA541787 | Hs.23837 | | Affymetrix | OA > RA |
| AA775711 | Hs.348392 | | Affymetrix | OA > RA |
| AI659927 | Hs.6634 | | Affymetrix | OA > RA |
| AI084224 | Hs.53542 | | Affymetrix | OA > RA |
| AI123555 | Hs.81796 | | Affymetrix | OA > RA |
| W73230 | Hs.7913 | | Affymetrix | OA > RA |
| W27376 | Hs.8395 | hypothetical protein | Affymetrix | OA > RA |
| | | FLJ10781 | | |
| AW022607 | Hs.12482 | glyceronephosphate O- | Affymetrix | OA > RA |
| | | acyltransferase | | |
| W70242 | Hs.58086 | | Affymetrix | OA > RA |
| W25528 | Hs.89319 | | Affymetrix | OA > RA |
| AA947123 | Hs.8861 | | Affymetrix | OA > RA |
| AA528821 | Hs.235857 | | Affymetrix | OA > RA |
| AA131648 | Hs.23767 | hypothetical protein | Affymetrix | OA > RA |
| | | FLJ12666 | | |
| R12398 | Hs.21075 | GTF2I repeat domain- | Affymetrix | OA > RA |
| | | containing 1, isoform 1 | | |
| | | NM_005685 | | |
| W52683 | Hs.107260 | hypothetical protein | Affymetrix | OA > RA |
| | | DKFZp586H0623 | | |
| W72194 | Hs.108924 | ponsin NM_015385 | Affymetrix | OA > RA |
| AA885516 | Hs.104627 | | Affymetrix | OA > RA |
| W68796 | Hs.237731 | | Affymetrix | OA > RA |
| AI879337 | Hs.323432 | mammalian inositol | Affymetrix | OA > RA |
| | | hexakisphosphate kinase 2 | | |
| W45581 | Hs.23133 | | Affymetrix | OA > RA |
| N98637 | Hs.7759 | | Affymetrix | OA > RA |
| AI809953 | Hs.123933 | | Affymetrix | OA > RA |
| T68423 | Hs.11873 | | Affymetrix | OA > RA |
| AL044670 | Hs.182364 | | Affymetrix | OA > RA |
| AA779895 | Hs.19339 | | Affymetrix | OA > RA |
| AI719167 | Hs.12731 | | Affymetrix | OA > RA |
| T99215 | Hs.168640 | ankylosis, progressive | Affymetrix | OA > RA |
| | | homolog NM_054027 | | |
| | | ankylosis, progressive | | |
| | | homolog | | |
| AA534296 | Hs.20953 | | Affymetrix | OA > RA |
| AI819043 | Hs.21342 | | Affymetrix | OA > RA |
| AI762879 | Hs.86437 | | Affymetrix | RA > OA |
| W61000 | Hs.238730 | | Affymetrix | RA > OA |
| AL043192 | Hs.103378 | | Affymetrix | RA > OA |
| AI741313 | Hs.103657 | | Affymetrix | RA > OA |
| AI031674 | Hs.236494 | ras-related GTP-binding | Affymetrix | RA > OA |
| | | protein | | |
| AA670193 | | | Affymetrix | RA > OA |
| AW005250 | Hs.238936 | | Affymetrix | RA > OA |
| AA682496 | Hs.270737 | tumor necrosis factor | Affymetrix | RA > OA |
| | | (ligand) superfamily, | | |
| | | member 13b | | |
| AI128225 | Hs.914 | | Affymetrix | RA > OA |
| AW026543 | Hs.238936 | | Affymetrix | RA > OA |
| AI991095 | Hs.293441 | | Affymetrix | RA > OA |
| AI872510 | Hs.181125 | | Affymetrix | RA > OA |
| AI828404 | Hs.300697 | | Affymetrix | RA > OA |
| AI807353 | Hs.237868 | interleukin 7 receptor | Affymetrix | RA > OA |
| AL048481 | Hs.11571 | | Affymetrix | RA > OA |
| AW014626 | Hs.10949 | | Affymetrix | RA > OA |
| AI400414 | | | Affymetrix | RA > OA |
| AI655112 | Hs.16179 | hypothetical protein | Affymetrix | RA > OA |
| | | FLJ23467 | | |
| AI936345 | Hs.95549 | hypothetical protein | Affymetrix | RA > OA |
| AI961907 | Hs.179573 | alpha 2 type I collagen | Affymetrix | RA > OA |
| | | preproprotein | | |
| AI743730 | Hs.30822 | hypothetical protein | Affymetrix | RA > OA |
| | | FLJ11110 | | |
| AI990512 | Hs.34192 | | Affymetrix | RA > OA |
| AI741715 | Hs.1466 | glycerol kinase | Affymetrix | RA > OA |
| T66305 | Hs.12920 | hypothetical protein | Affymetrix | RA > OA |
| | | FLJ20668 | | |
| AA424160 | Hs.165909 | | Affymetrix | RA > OA |
| AI075407 | Hs.296083 | | Affymetrix | RA > OA |
| AA811088 | Hs.24143 | WASP-interacting protein | Affymetrix | RA > OA |
| AI978918 | Hs.179608 | retinol dehydrogenase | Affymetrix | RA > OA |
| | | homolog | | |
| AA740831 | Hs.193514 | | Affymetrix | RA > OA |
| W84421 | Hs.349096 | | Affymetrix | RA > OA |
| AA233208 | Hs.91165 | hypothetical protein | Affymetrix | RA > OA |
| AA886976 | Hs.95821 | osteoclast stimulating | Affymetrix | RA > OA |
| | | factor 1 | | |
| AA864400 | Hs.71215 | docking protein 2, 56 kD | Affymetrix | RA > OA |
| AI073984 | Hs.14453 | interferon consensus | Affymetrix | RA > OA |
| | | sequence binding protein 1 | | |
| AI983633 | Hs.179573 | alpha 2 type I collagen | Affymetrix | RA > OA |
| | | preproprotein | | |
| AI564488 | Hs.300697 | | Affymetrix | RA > OA |
| AI655781 | Hs.237868 | interleukin 7 receptor | Affymetrix | RA > OA |
| AA814195 | Hs.184465 | hypothetical protein | Affymetrix | RA > OA |
| | | FLJ11259 | | |
| AI916783 | Hs.234149 | hypothetical protein | Affymetrix | RA > OA |
| | | FLJ20647 | | |
| AA829355 | Hs.267993 | hypothetical protein | Affymetrix | RA > OA |
| | | FLJ10143 | | |
| N66595 | Hs.24283 | | Affymetrix | RA > OA |
| AA165400 | Hs.10927 | | Affymetrix | RA > OA |
| AI478759 | Hs.234149 | hypothetical protein | Affymetrix | RA > OA |
| | | FLJ20647 | | |
| AI655719 | Hs.2157 | Wiskott-Aldrich | Affymetrix | RA > OA |
| | | syndrome protein | | |
| N63815 | Hs.110121 | SEC7 homolog | Affymetrix | RA > OA |
| AW001184 | Hs.44672 | hypothetical protein | Affymetrix | RA > OA |
| | | FLJ10470 | | |
| N21390 | Hs.5888 | | Affymetrix | RA > OA |
| AA587944 | Hs.259737 | FN5 protein | Affymetrix | RA > OA |
| AI951459 | Hs.7337 | hypothetical protein | Affymetrix | RA > OA |
| | | FLJ10936 | | |
| AA464464 | Hs.10949 | | Affymetrix | RA > OA |
| AI692538 | Hs.11135 | | Affymetrix | RA > OA |
| AI817147 | Hs.181301 | cathepsin S | Affymetrix | RA > OA |
| AI263085 | Hs.17914 | CD20-like precusor | Affymetrix | RA > OA |
| W58252 | Hs.182793 | golgi phosphoprotein 2 | Affymetrix | RA > OA |
| AA056180 | Hs.70704 | | Affymetrix | RA > OA |
| AA224174 | Hs.111099 | | Affymetrix | OA > RA |
| AI571452 | Hs.11169 | Gene 33 | Affymetrix | OA > RA |
| AA155952 | Hs.349303 | | Affymetrix | OA > RA |
| W68504 | Hs.191098 | | Affymetrix | OA > RA |
| AI200456 | Hs.48516 | | Affymetrix | OA > RA |
| AW003093 | Hs.349764 | | Affymetrix | OA > RA |
| AI190027 | Hs.38034 | | Affymetrix | OA > RA |
| R52934 | Hs.8562 | hypothetical protein | Affymetrix | OA > RA |
| | | FLJ20374 | | |
| W44633 | Hs.301296 | | Affymetrix | OA > RA |
| AW024474 | Hs.44276 | homeo box C10 | Affymetrix | OA > RA |
| AI806502 | Hs.334800 | | Affymetrix | OA > RA |
| AI492370 | Hs.105606 | hypothetical protein | Affymetrix | OA > RA |
| | | FLJ20512 | | |
| AW021179 | Hs.90443 | NADH dehydrogenase | Affymetrix | OA > RA |
| | | (ubiquinone) Fe—S protein | | |
| | | 8 (23 kD) (NADH- | | |
| | | coenzyme Q reductase | | |
| AI679110 | Hs.323067 | | Affymetrix | OA > RA |
| R85633 | | | Affymetrix | OA > RA |
| N91161 | Hs.117176 | poly(A)-binding protein, | Affymetrix | OA > RA |
| | | nuclear 1 | | |
| AW020657 | | | Affymetrix | OA > RA |
| AI871043 | Hs.173233 | hypothetical protein | Affymetrix | OA > RA |
| | | FLJ10970 | | |
| N39237 | Hs.44977 | | Affymetrix | OA > RA |
| AI949833 | Hs.21914 | | Affymetrix | OA > RA |
| AA679297 | Hs.109494 | secreted protein of | Affymetrix | OA > RA |
| | | unknown function | | |
| AI962647 | Hs.182364 | | Affymetrix | OA > RA |
| AL037611 | Hs.285902 | | Affymetrix | OA > RA |
| AI871278 | Hs.301804 | | Affymetrix | OA > RA |
| AI357650 | Hs.28847 | AD026 protein | Affymetrix | OA > RA |
| AI149793 | Hs.38034 | | Affymetrix | OA > RA |
| AI797684 | Hs.39619 | hypothetical protein | Affymetrix | OA > RA |
| | | LOC57333 | | |
| R52250 | Hs.348297 | | Affymetrix | OA > RA |
| AI669738 | Hs.128856 | CSR1 protein | Affymetrix | OA > RA |
| AA058770 | Hs.18987 | | Affymetrix | OA > RA |
| AI039005 | Hs.164680 | | Affymetrix | OA > RA |
| AI936560 | Hs.6136 | | Affymetrix | OA > RA |
| AA521373 | Hs.9469 | pleckstrin homology | Affymetrix | OA > RA |
| | | domain-containing, family | | |
| | | A (phosphoinositide | | |
| | | binding specif | | |
| H15888 | Hs.27621 | sema domain, seven | Affymetrix | OA > RA |
| | | thrombospondin repeats | | |
| | | (type 1 and type 1-like), | | |
| | | transmembran | | |
| AI333793 | Hs.337062 | | Affymetrix | OA > RA |
| AA523172 | Hs.103135 | | Affymetrix | OA > RA |
| AI860960 | Hs.352081 | | Affymetrix | OA > RA |
| AI355848 | Hs.35841 | nuclear factor I | Affymetrix | OA > RA |
| AI982754 | Hs.75106 | clusterin (complement | Affymetrix | OA > RA |
| | | lysis inhibitor, SP-40, 40, | | |
| | | sulfated glycoprotein 2, | | |
| | | testos | | |
| AI800218 | Hs.289019 | latent transforming | Affymetrix | OA > RA |
| | | growth factor beta binding | | |
| | | protein 3 | | |
| AW016356 | Hs.126857 | | Affymetrix | OA > RA |
| AA968552 | Hs.25523 | | Affymetrix | OA > RA |
| AI634557 | Hs.28107 | | Affymetrix | OA > RA |
| AW025494 | Hs.95867 | hypothetical protein | Affymetrix | OA > RA |
| | | EST00098 | | |
| AA628405 | Hs.339352 | | Affymetrix | OA > RA |
| AI810399 | Hs.55940 | | Affymetrix | OA > RA |
| AA029735 | Hs.159993 | | Affymetrix | OA > RA |
| AA723927 | Hs.209569 | | Affymetrix | OA > RA |
| AI799784 | Hs.49696 | | Affymetrix | OA > RA |
| AI817330 | Hs.110477 | dolichyl-phosphate | Affymetrix | OA > RA |
| | | mannosyltransferase | | |
| | | polypeptide 3 | | |
| AI990803 | Hs.293782 | | Affymetrix | OA > RA |
| AA034418 | Hs.30627 | | Affymetrix | OA > RA |
| AA115295 | Hs.284208 | DKFZP434N161 protein | Affymetrix | OA > RA |
| AI673281 | Hs.181444 | hypothetical protein | Affymetrix | OA > RA |
| W63805 | Hs.84344 | CGI-135 protein | Affymetrix | OA > RA |
| AA427597 | | TGFβ-induc early growth | Unigene | NS > RA |
| | | response 2 | | |
| AA806239 | | IG-ALPHA2-C REGION | Unigene | RA > NS |
| AB014518 | | KIAA0618 | Unigene | RA > NS |
| AB021871 | | AK1 | RDA, Unigene | RA > OA, |
| | | | | RA > NS |
| AF 000984 | | DBY altern transcript 2 | Affymetrix | NS > RA |
| AF 001691 | | cornified envelope | Affymetrix | NS > RA |
| | | precursor | | |
| AF005058 | | CXC | | |
| AF0605668 | | leukemia zink finger | Affymetrix | OA > RA |
| | | PLZF | | |
| AF068293 | | HDCMB07P/PCM-1 | Unigene | RA > NS |
| AF105036 | | GKLF | RDA | OA > NS |
| AF182035 | | a Actin | RDA | OA > NS |
| AF182035 | | myosin light chain | RDA | OA > NS |
| AF216292 | | BIP | | |
| AF218004 | | CSNK1A1 | Unigene | RA > NS |
| AJ000542 | | natural killer cell receptor | RDA | RA > OA |
| | | p58 | | |
| J05008 | | EDN1 | Affymetrix | NS > RA |
| L08187 | | cytokine receptor EBI 3 | RDA | RA > OA |
| L31581 | | EBI1/CCR7 | Affy | RA > NS |
| L37036 | | ENA-78 | =Affymetrix | RA > OA |
| M10988 | | TNFμ | | |
| M19997 | | elongation factor 2 | RDA | RA > OA |
| M29469 | | Ig rearranged k chain (VJ | RDA, | RA > OA |
| | | regions) | Affymetrix | |
| M31164 | | TSG6 | RDA, Unigene | RA > OA, |
| | | | | RA > NS |
| M83248 | | OSTP (Osteopontin) | RDA, | RA > OA |
| | | | Affymetrix | |
| NM_002450 | | Metallomethionein | Unigene | NS > RA |
| NM_003573 | | TGFβ-BP4 | Unigene | RA > NS |
| NM_000362 | | TIMP-3 | RDA | NS > RA |
| NM_000396 | | Cathepsin K | RDA | RA > OA, |
| | | | | OA > NS |
| NM_0006091 | | SDF1 | RDA | OA > NS |
| NM_001908 | | Cathepsin B | RDA | OA > NS |
| NM_002084 | | glutathion peroxidase 3 | RDA | NS > RA |
| NM_002229 | | Jun B | Unigene | NS > RA |
| NM_002989 | | SLC | Unigene | RA > NS |
| NM_003966 | | SEMA5A | RDA | RA > OA |
| NM_004039 | | Annexin II | RDA | RA > OA, |
| | | | | OA > NS |
| NM_005368 | | Myoglobin | RDA | OA > NS |
| NM_006472 | | VDUP1 | RDA, Unigene | NS > RA |
| NM_007016 | | Mysin light polypeptid2 | RDA | OA > NS |
| NM_015675 | | GADD45B/MYD118 | RDA, Unigene | NS > RA |
| R75775 | | EGR1 | Unigene | NS > RA |
| U070136 | | megakaryocyte | RDA, Unigene | NS > RA |
| | | stimulating factor | | |
| U34690 | | CORO1A/p57 | Unigene | RA > NS |
| U93569 | | L1 element | RDA, Unigene | RA > OA; |
| | | | | RA > NS |
| X03754 | | SCYA3 (MIP a)/GOS19 | Unigene | RA > NS |
| X0523 | | MMP1 | | |
| X14723 | | Clustrin/SP40 | RDA, Unigene | NS > RA |
| X15332 | | collagen III a1 | RDA, Unigene | RA > OA |
| X54629 | | c-myc | RDA, Unigene | NS > RA |
| X54629 | | pHL-1 gene | RDA | NS > RA |
| X58122 | | Nebulin | RDA | OA > NS |
| X62996 | | mitochondrial mRNA | RDA | OA > NS |
| X63596 | | TRE-2 | RDA | RA > OA |
| X65968 | | PMP22 | Unigene | RA > NS |
| X88971 | | HLA DRB1 | RDA | RA > OA |
| X94771 | | EMP3 | Unigene | RA > NS |
| XM 008868 | | latent transforming | RDA, Unigene | NS > RA |
| | | growth factor beta binding | | |
| | | prot. LTBP4 | | |
| XM_031289 | | interleukin 8 | =Affymetrix | RA > OA |
| XM012651 | | collagen I a1 | RDA | RA > OA |

Combination of the Proteins

TABLE 2

| | Example for |
|---|---|
| Proteine | accession |
| 78 kDa glucose-regulated protein precursor (GRP 78) (Immunoglobulin | P11021 |
| heavy chain binding protein) (BIP) (Endoplasmic reticulum lumenal Ca2+ | |
| binding protein grp78) | |
| Citrullinierte Peptide (Peptids containing the deiminated form of Arginin | |
| [Citrullin]) | |
| Sa-Antigen | |
| RA33/Heterogeneous nuclear ribonucleoproteins A2/B1 (hnRNP A2/ | P22626 |
| hnRNP B1) | |
| Calpain inhibitor (Calpastatin) (Sperm BS-17 component) | P20810 |
| Calreticulin precursor (CRP55) (Calregulin) (HACBP) (ERp60) | P27797 |
| Synovial stimulatory protein P205 | P80697 |
| Filaggrin precursor | P20930 |
| Fibrin | |
| Fibrinogen alpha/alpha-E chain precursor [Contains: Fibrinopeptide A] | P02671 |
| Fibrinogen beta chain precursor [Contains: Fibrinopeptide B] | P02675 |
| Fibrinogen gamma chain precursor (PRO2061) | P02679 |
| DnaJ | |
| Ig gamma-1 chain C region | P01857 |
| Ig gamma-2 chain C region | P01859 |
| Ig gamma-3 chain C region (Heavy chain disease protein) (HDC) | P01860 |
| Ig gamma-4 chain C region | P01861 |
| 60 kDa heat shock protein, mitochondrial precursor (Hsp60) (60 kDa | P10809 |
| chaperonin) (CPN60) (Heat shock protein 60) (HSP-60) (Mitochondrial | |
| matrix protein P1) (P60 lymphocyte protein) (HuCHA60) | |
| EBNA-1 NUCLEAR PROTEIN | P03211 |
| IR-3, Internal Repeat Region (in EBNA-1 e.g. Proteins) | |
| Chitinase-3 like protein 1 precursor (Cartilage glycoprotein-39) (GP-39) (39 kDa | P36222 |
| synovial protein) (YKL-40) | |
| Collagen alpha 1(II) chain precursor [Contains: Chondrocalcin] | P02458 |
| CH65, Chondrocyte Antigene 65 | |
| Collagen-binding protein 2 precursor (Colligin 2) (Rheumatoid arthritis | P50454 |
| related antigen RA-A47) | |
| 47 kDa heat shock protein precursor (Collagen-binding protein 1) (Colligin | P29043 |
| 1) | |
| Chitinase 3-like protein 2 precursor (YKL-39) (Chondrocyte protein 39) | Q15782 |
| Chitinase 3-like protein 2 precursor (YKL-39) (Chondrocyte protein 39) | Q15783 |
| Chitinase 3-like protein 2 precursor (YKL-39) (Chondrocyte protein 39) | Q15749 |
| Fructose-bisphosphate aldolase A (Muscle-type aldolase) (Lung cancer | P04075 |
| antigen NY-LU-1) | |
| Proteoglycan link protein precursor (Cartilage link protein) (LP) | P10915 |
| Matrix metalloproteinase-19 precursor (MMP-19) (Matrix | Q99542 |
| metalloproteinase RASI) | |
| MMP-19 (matrix metalloproteinase) | CAA63299 |
| Aggrecan core protein precursor (Cartilage-specific proteoglycan core | P16112 |
| protein) (CSPCP) (Chondroitin sulfate proteoglycan core protein 1) | |
| Ezrin (p81) (Cytovillin) (Villin 2) | P15311 |
| Radixin | P35241 |
| Moesin (Membrane-organizing extension spike protein) | P26038 |

The invention will now be described by means of examples, however without being limited to them.

### EXAMPLES

### Example 1

### Employment in Clinical Diagnostics

A patient, having articular symptoms for 4 month, suffers from an asymmetric swelling and painfulness in 2 proximal joints and 1 middle joint of the finger and in the right wrist joint. The stiffnless in the morning persists for about 30 minutes. The radiological picture shows a beginning erosive alteration in one proximal joint of the toe. The C-reactive Protein is within the normal range, the sedimentation rate is slightly increased, rheumatoid factor and HLA-DR4 are negative. There is no familiar history concerning an inflammatory rheumatoid disease.

During an ambulant appointment, a synovial biopsy from the right wrist joint was isolated by minimally invasive arthroscopy. Of four samples having a weight of a about 10 mg each, a little sample is fixed in formalin for the following histological evaluation. The remaining samples are introduced into RNA lysis-buffer, crushed up and the RNA is extracted according to standard protocols. After the (reverse) transcription into cDNA, an in vitro transcription into a biotin-labelled cRNA constituting a transcription of the cDNA, is performed. The cRNA is fragmented and then employed for the hybridisation to the DNA-array.

The array is produced by a commercial company for the generation of DNA-arrays, like e.g. Affymetrix. There, suitable oligonucleotides are deduced from the sequences of table 1 and from the gene sequences coding for the proteins of table 2, whereat these oligonucleotides allow for a specific hybridisation to the respective cRNA-sequences. These sequences are either synthesized as oligonucleotides and then printed onto an array-carrier, or they are directly synthesized on the carrier, e.g. by a photolithographic method.

The hybridisation is performed according to the instructions of the manufacturer's protocol. The DNA-array is read by means of a scanner. The translation of the optical information into expression signals is accomplished by using standard software, like e.g. “Micro-Array Suite” from Affymetrix. One now has obtained the signals of the RNA expression rates of the genes or proteins mentioned in the tables 1 and 2. Starting from this newly defined selection of genes for the diagnostic evaluation and therapy development for joint diseases, clinically and histologically characterized tissue samples were classified and related to each other in a hierarchical manner after cluster analysis during preliminary tests. Due to the comparative association with the clinical findings, this classification was accomplished in particular in dependence on the type of disease (arthrosis, reactive arthritis, rheumatoid arthritis, subgroups of rheumatoid arthritis), the activity of the disease und thus the prognosis and the possibility of affecting the pathologically altered gene expression by means of an applied drug. The signal data of the above mentioned patient are then compared to this database. Thereby, an assignment to one of these groups becomes possible, and one can obtain information about the corresponding clinical associations. Thus, one obtains evidence about the diagnosis, the activity, the prognosis and the therapeutic options in the individual patient.

### Example 2

### Employment for the Evaluation of Therapies

A patient, who has been suffering from a chronic joint inflammation for 5 years, diagnosed as a rheumatoid arthritis, shows progressive specific radiological changes in several fmger joints, accompanied by pain and swelling in several finger joints, the left elbow joint and the right ankle joint despite a current basal therapy under application of 15 mg of Methotrexate per week. During an ambulant appointment, a synovial biopsy from the left elbow joint was isolated by minimally invasive arthroscopy. Several samples of about 30 mg total weight were introduced into lysis-buffer, crushed up and the RNA was extracted. The preparation of the sample was accomplished in a similar manner as in example 1. The same DNA chip like in example 1 is used for analysis. After hybridisation, the transfer of the results of hybridisation into a picture data file and translation of the results into signal information for each of the tested genes, an assignment to defined expression pattern is accomplished. These patterns were determined in preliminary tests, thereby using the defined selection of genes from table 1 and 2 being newly defined in this specification. Thereby, the alteration of the expression profile of a sample was analysed in dependence on the respective joint disease, which is affected by defined drugs applied in defined concentrations. The profiles were hierarchically classified, thereby considering the association with the employed drugs and the applied dose. When the patient sample is compared to these defined expression patterns, the assignment to a specific pattern and the therapeutic efficiency information associated therewith make it possible to estimate, if the applied drug Methotrexate could be effective at a higher dose, or if it is reasonable to change to a drug, the activity profile of which fits best for affecting the pathological changes in the individual case.

### Example 3

### Autoreactivity Profiles in the RA

The RA is different from other rheumatic and other inflammatory diseases in respect of the generation of auto-antibodies. Thereby, a distinction between RA and non-RA is not provided by one antibody-reactivity, but by different profiles of several autoreactivities. It is thus possible to obtain save diagnostics, to control therapeutic progress and to perform preventive examinations based on the determination of the RA-specific autoreactivity profiles.

Antibodies are directed against antigens, or, more precisely, against epitopes, which are bound by the paratopes during a specific antibody-antigen-reaction. An epitope is defined as the region of an antigen, which specifically interacts with an antibody (i.e. with its paratope). In general, an epitope is understood as a peptide sequence of a protein, whereat this peptide sequences comprises about 16 to 20 amino acids. This sequence can be consecutive (continuous epitope) or interrupted (discontinuous epitope). Typically however, there are only a few amino acids, in rare cases just one amino acid, necessary and sufficient for the specific interaction between antibody and antigen. Meanwhile, it is known, that even nucleic acids can act as antigens. Particular importance is more and more attributed to posttranslational modifications like e.g. phosphorylation, acylation, glycosylation, methylation, deimination, etc. Since these modifications often have a regulatory function, they seem to be particularly interesting as target structures of antigens, especially under pathological conditions. Since it has already been shown for some RA-associated auto-antigens, that specific post-translational modifications produce epitopes for auto-antibodies, it has to be paid particular attention thereupon, that these structures are realized in the test system.

The proteins listed in table 2 have been described as RA-associated auto-antigens. The relevance of most of these single components however, is low or not obvious for the diagnostics of RA. The same applies to the genes being overexpressed on the MRNA level, which are listed in table 1. These components by themselves are not suitable to significantly improve the diagnostics of RA. This is shown by the fact, that practically the majority of the proteins listed in tables 1 and 2 are not applied for as patents for this respective purpose. Only a few proteins are such characteristic, that a relevance for RA has been assumed. This is e.g. valid for the protein BiP (Heavy Chain Binding Protein), which is the target of an immune reaction in RA. Here, e.g. a post-translational modification in the form of a glycosylation has to be taken into account, since this modification is a component of epitopes, which are both necessary for the recognition of auto-antibodies in RA, and for the distinction between RA- and non-RA-auto-antibodies. Moreover, the amino acid being post-translationally transformed from arginine to citrullin was described as an essential epitope for RA-associated auto-antibodies (6). A similarly high significance for the diagnostics of RA is valid for the Sa-antigen (5), the RA33-antigen and for Calpastatin.

Nevertheless, these components by themselves were not appropriate to allow for an unambiguous diagnosis of RA or even for the monitoring of a therapy. The depicted, novel approach according to the invention refers to the immunome of RA. The immunome of RA comprises the entirety of autoreactive antibodies, which are present in RA, and also the entirety of the auto-antigens or auto-epitopes recognized by these antibodies. Unexpectedly, it was able to be found, that it is possible for the first time to diagnose a disease unambiguously as an RA by analysing the combination of RA-associated auto-antibodies. It was able to be shown for the first time, that there exist different patterns of auto-antibodies, which exclusively occur in RA. These patterns also include such auto-antigens or autoreactivities, which by themselves appear to be unimportant for the RA. These is even more surprising, since respective first approaches of other groups did not lead to this finding, although it is emphasized, that the most important auto-antigens from eight different human autoimmune diseases were employed (11). The same applies for an approach, in which auto-antigens were used, which are relevant for another rheumatic disease, the systemic Lupus erythematodes (SLE). Apparently, the essential difference between the approaches already being published and the approach described herein, is on the one hand based on the type of analysis (multivariate), on the other hand on the composition of the auto-antigens. Only a sufficiently high number of RA-relevant auto-reactivities allows for an unambiguous diagnosis. Thus, the entirety of the RA-associated auto-antibodies and auto-antigens constitutes information, which—together with other techniques (protein array technology (27), data processing)—can be, among other applications, employed as a means for the diagnostics and classification of RA. Even an expert in this field would not have been able to conclude such a use degree by means of analogy deduction. The immunome of RA and also mere parts of the RA-immunome can be employed for unambiguously distinguishing RA from other diseases or from the healthy state. A commercial utilization of the unexpected invention moreover only becomes possible by the currently available or still developing possibilities of the high-throughput technologies. This refers in particular to the multiple-parameter-analysis of autoreactivities, since it is necessary in this place, to perform a multiplicity of parallel analyses under the employment of miniscule sample sizes derived from the patient.

Proteins or partial protein sequences of the components given in table 2, or proteins and partial protein sequences encoded by genes given in table 1, including the post-translational modifications being potentially necessary for the distinction between RA and non-RA, are synthesized and provided for the generation of autoreactivity profiles. The synthesis can be accomplished by an arbitrary, known approach based on molecular biology or by an arbitrary approach of protein chemistry. Furthermore, partially artificial (in vitro translation) or artificial synthesis according to the state of the art are suitable to produce said proteins or partial protein sequences.

Protein Array/Peptide Array (28)

Proteins or partial protein sequences according to table 2 or 1 are used in their entirety or only as a respective selection suitable for the immunomic distinction of clinical pictures, in order to create a test option, which is suitable to determine the autoreactivities of an individual. This particularly refers to the selection of Citrullin, BiP, p205, IgG, Calpastatin, RA33, Sa-antigen and Calreticulin. For this aim, the proteins are separately applied to a carrier matrix at positions allowing for a spatial resolution. The position and identity of each immobilized protein, peptide, modified protein or modified peptide are known. The micro-format allows for a parallel detection of thousands of different antigens and/or auto-antigens (proteins/peptides) in the sub-microliter range of human sera. Preferred options are the preparation of a Protein Array, of a high-density filter, of a high-density glass carrier or of another matrix produced by the high-density method, whereat this matrix in a coated or non-coated form is coupled to proteins or partial protein sequences. For instance, proteins or partial protein sequences can be printed onto derivatized or coated/activated glass carriers, or the application is accomplished by means of the ink jet-method, in a capillary manner, or by direct synthesis on the array under the employment of photolithographic masks or digital micro-reflectors. Instead of glass carriers one can also use membranes and filters, polystyrene matrices, Nanowell-plates and micro-particles (29).

The Protein Array is incubated together with a suitable dilution of patient sera or as well of patients' joint effusions. During this incubation, possibly present antibodies having specificity for one or several protein components can bind to these protein-antigens. This is followed by a washing step in order to remove remaining free antibody and serum components. Then one incubates the sample with a second antibody, which is suitable both to indicate a successful antigen-antibody-reaction by binding the first antibody and to introduce a suitable label, which allows for visualization and quantification, suitably a covalently coupled fluorescence dye or a covalently coupled enzyme being capable to produce a dye from a precursor substance. This is followed by a further washing step in order to remove the remaining free second antibody.

Suspension Array (30)

The Suspension Array uses plastic particles as a matrix, whereat the plastic particles are coated with the mentioned proteins. This is such accomplished, that the optical characteristics of particles coupled to a specific protein are different to the optical characteristics of particles coupled to another protein. The imnuunomic analysis is performed in an analogous manner by the incubation with patient sera or other bodily fluids. By means of the antibody-reaction with a suitable second antibody, a further optical (fluorescent) signal is produced either directly or again indirectly. The analysis is then performed in a multicolor-fluorescence activated cell (FAC-) scan.

Time-Resolved Protein Arrays (31)

A polystyrene surface is coupled to different proteins or partial protein sequences taken from table 1 and 2. The antibodies to be analysed from the patient sera are biotinylated by using an active biotin-ester. Alternatively, one may also use biotinylated secondary antibodies being specific for human antibodies in order to avoid inter-patient-deviations in consequence of a different efficiency of biotinylation. The patient antibodies are then incubated with the protein-coupled polystyrene surface. After a subsequent washing step, the detection is accomplished by means of Streptavidin, which is coupled to a fluorescent Europium complex. The evaluation is then accomplished after a washing and drying step by means of laser-excited, time-resolved solid phase fluorescence analysis.

Data Patterns and Multifactor Analysis

Parameters (e.g. the autoreactivities obtained for the proteins/auto-antigens listed in tables 1 and 2; e.g. the autoreactivities RF/Citrullin/BiP/Calpastatin/Calreticulin/RA33) are determined as complete as possible. Data patterns of individual patients having more than 2 of 6 missing values were a priori excluded from the analysis.

The interpretation of the irrmunodetection system yields a negative or positive result for each patient and each auto-reactivity. An alternative option are continuous values (Protein Array, ELISA), which are divided into positive or negative either artificially (mathematically) or by a control group-related Cut Off (analysis in comparison to a suitable control group, e.g. age- and sex-matched healthy controls or control-patients suffering from another disease). Each data pattern is analysed and classified by means of the CLASSIF1 program system (32).

In a first step, the triple-matrix characters of each clinical diagnosis category are entered into the first reference classification mask. Each patient is then classified according to the highest degree of position identity between the patient mask and a clinical reference mask.

In a second step, those data columns are eliminated, which display the triple-matrix character “0” for all reference masks, since they do not allow for a distinction between the disease entities.

In a third step, the CLASSIPF1-algorithm transiently eliminates either individual parameters or combinations of two parameters in all permutations from the classification process. The total data set is then reclassified. Parameters, which affect the classification result by their transient elimination, are informative, since obviously no essential information is lost. The information content of each parameter is intermittently provided by the algorithm, reintroduced after the operation and the next parameter or the next pair of parameters is transiently extracted and analysed in an analogous manner. The intermittent removal and reintroduction is performed, until the information content of all parameters, either alone or in combination, is revealed. Parameters, which prove to be uninformative either alone or in combination with a further parameter, are eliminated. The remaining sequence of informative parameters constitutes the reference classification mask for the respective clinical prediction category.

In a fourth step, the classification is optimized by classifying the percentile Cut Off values 10/90%, 15/85%, 20/80%, 25/75% and 30/70% with the subsequent selection of the pair showing the best discriminating properties. The best classification results are typically reached in the range between the 10/90% and 25/75% percentile pairs. Negative and positive predictive values in a Confusion Matrix provide information about how good the reference sample and the samples to be tested are discriminated by the employed pattem(s). Additionally, the data patterns of each patient are subjected to a multifactor analysis. The multifactors for five parameter patterns were obtained by multiplication or division of the different parameters in all possible combinations, followed by the standardization of the five data columns towards the mean values of the RA-reference group. Subsequently, the mean values for each parameter of the other patient groups (e.g. OA, reA, PsoA, other) were determined. Multifactors for all parameter permutations were either determined by multiplication, when the parameter's mean value of the respective patient group was increased in comparison to the reference value (RA), or by division, when the value was decreased.

The multifactor database comprises the measured parameters (RF/Citrullin/BiP/Calpastatin/Calreticulin/RA33). 26 multifactors have been classified via the CLASSIF1-algorithm. Thereby, all figures of each database column were transformed either into “−” (less than the lower percentile of the value distribution of the reference patients [RA]), “0” (between the lower and upper percentile) or “+” (larger than the upper percentile) triple-matrix characters. Following the transformation of the database columns, a confusion matrix is established between clinical diagnosis and computer classification.

The diagonal values of this confusion matrix represent the specificity of the reference samples and the sensitivity of the samples to be tested. These are further optimized during the subsequent iterative learning process. An optimal classification is achieved, when all samples have been correctly classified, that is when all diagonal values of the confusion matrix reach 100% and the values of the non-diagonal fields are 0%. The learning process serves to eliminate non-informative parameters and thus to accumulate the discriminating parameters.

### DESCRIPTION OF THE FIGURE

FIG. 1: Autoreactivity pattern with RA33, RiF, Citrullin, BiP and Calpastatin

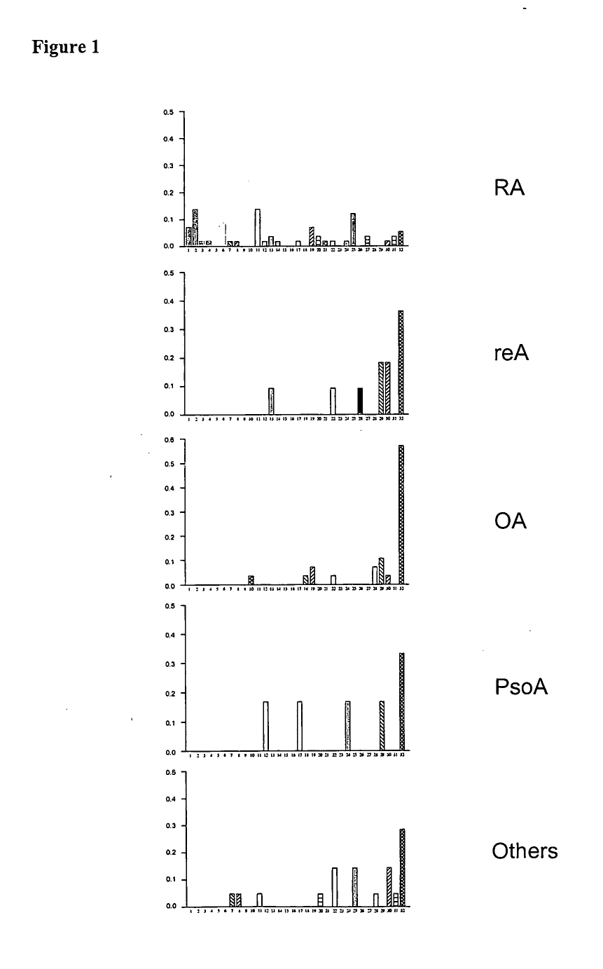

Depicted are all 32 possible combinations of the autoreactivities against IgG (RF), Citrullin, BiP, Calpastatin, RA33 and Calpastatin for the disease entities RA (rheumatoid arthritis), reA (reactive arthritis), OA (osteoarthrosis), PsoA (psoriasis-associated arthritis) and other.

### LIST OF ABBREVIATIONS

ACR American College of Rheumatology
BiP Binding Protein, Heavy Chain Binding Protein
BSA Bovine Serum Albumin
Calp Calpastatin
Calr Calreticulin
cDNA complementary DNA, copy DNA
CH Chondrocyte Antigen
Cit citrullinated peptide
CrP C-reactive Protein
DNA desoxyribonucleic acid
DPNII from Diplococcus pneumoniae
dNTP desoxynucleotide-triphosphates (equimolar mixture of dATP, dCTP,
dGTP, dTTP)
dNTP desoxynucleotide-triphosphate
EBNA-1 Epstein-Barr virus nuclear antigen-I
EBV Epstein-Barr virus
ER endoplasmatic reticulum
FACS Fluorescence Activated Cell Sorting
GAPDH Glycerol-aldehyde-phosphate dehydrogenase
HC Human Cartilage
HC gp39 Human Cartilage glycoprotein 39
HLA-system histocompatibility antigen (HLA—human leucocyte antigen)
HLA-DR4 HLA feature, that exhibits an increased association with a rheumatoid arthritis
hnRNP heterogeneous ribonucleoprotein (RA33)
Hsp Heat shock protein
Ig immunoglobulin
IgG immunoglobulin G
IL- interleukin
IR-3 internal repeat region 3
MCTD Mixed Connective Tissue Disease (mixed collagenosis)
MHC- Major Histocompatibility Complex
MMP matrix metalloproteinase
mRNA messenger ribonucleic acid
NAD nicotineamide-adenine-dinucleotide
NCBI National Centre for Biotechnology Information
ND normal donor
OA osteoarthrosis
O-GlcNAc O—N-acetylglucosamine
PCR Polymerase Chain Reaction
PHA phytohemagglutinin
PM/DM polymyositis/dermatomyositis
PsoA psoriasis-associated arthritis
RA rheumatoid arthritis
RA-A47 arthritis-related antigen
RA33 hnRNP A2
RDA Representational Difference Analysis
reA reactive arthritis
RF rheumatoid factors
RNA ribonucleic acid
RPMI commercially available, conventional cell culture medium, dilution Medium RPMI 1640; (Moore, G.E. et al., J. Am. Assoc. 199, 519-524, 1967)
RsaI DNA restriction enzyme RsaI from Rhodopseudomonas sphaeroides
RT Reverse Transcriptase (RT)
Sa-antigen 50 k-protein from human spleen and placenta
SLE systemic Lupus erythematodes
SSH Suppression Subtractive Hybridisation
TGF Transforming Growth Factor
UNIGENE UniGene is an experimental system for the automatic partition of the GeneBank-sequences into a non-redundant set of gene-orientated Clusters
YKL-39 Human Cartilage Related Protein

### REFERENCES

1. Arnett, F. C., S. M. Edworthy, D. A. Bloch, D. J. McShane, J. F. Fries, N. S. Cooper, L. A. Healey, S. R. Kaplan, M. H. Liang, H. S. Luthra, and et al. 1988. The American Rheumatism Association 1987 revised criteria for the classification of rheumatoid arthritis. Arthritis Rheum 31:315.
2. Kaps, C., C. Bramlage, H. Smolian, A. Haisch, U. Ungethum, G. R. Burmester, M. Sittinger, G. Gross, and T. Haupl. 2002. Bone morphogenetic proteins promote cartilage differentiation and protect engineered artificial cartilage from fibroblast invasion and destruction. Arthritis Rheum 46:149.
3. Roudier, J., G. Rhodes, J. Petersen, J. H. Vaughan, and D. A. Carson. 1988. The Epstein-Barr virus glycoprotein gp 110, a molecular link between HLA DR4, HLA DRl, and rheumatoid arthritis. Scand J Immunol 27:367.
4. Albani, S., E. C. Keystone, J. L. Nelson, W. E. Ollier, A. La Cava, A. C. Montemayor, D. A. Weber, C. Montecucco, A. Martini, and D. A. Carson. 1995. Positive selection in autoimmunity: abnormal immune responses to a bacterial dnaJ antigenic determinant in patients with early rheumatoid arthritis. Nat Med 1:448.
5. Despres, N., G. Boire, F. J. Lopez-Longo, and H. A. Menard. 1994. The Sa system: a novel antigen-antibody system specific for rheumatoid arthritis. J Rheumatol 21:1027.
6. Schellekens, G. A., B. A. de Jong, F. H. van den Hoogen, L. B. van de Putte, and W. J. van Venrooij. 1998. Citrulline is an essential constituent of antigenic determinants recognized by rheumatoid arthritis-specific autoantibodies. J Clin Invest 101:273.
7. Girbal-Neuhauser, E., J. J. Durieux, M. Amaud, P. Dalbon, M. Sebbag, C. Vincent, M. Simon, T. Senshu, C. Masson-Bessiere, C. Jolivet-Reynaud, M. Jolivet, and G. Serre. 1999. The epitopes targeted by the rheumatoid arthritis-associated antifilaggrin autoantibodies are posttranslationally generated on various sites of (pro)filaggrin by deimination of arginine residues. J Immunol 162:585.
8. Alsalameh, S., J. Mollenhauer, N. Hain, K. P. Stock, J. R. Kalden, and G. R. Burmester. 1990. Cellular immune response toward human articular chondrocytes. T cell reactivities against chondrocyte and fibroblast membranes in destructive joint diseases. Arthritis Rheum 33:1477.
9. DeRisi, J., L. Penland, P. O. Brown, M. L. Bittner, P. S. Meltzer, M. Ray, Y. Chen, Y. A. Su, and J. M. Trent. 1996. Use of a cDNA microarray to analyse gene expression patterns in human cancer. Nat Genet 14:457.
10. Haab, B. B., M. J. Dunham, and P. O. Brown. 2001. Protein microarrays for highly parallel detection and quantitation of specific proteins and antibodies in complex solutions. Genome Biol 2:RESEARCH0004.
11. Robinson, W. H., C. DiGennaro, W. Hueber, B. B. Haab, M. Kamachi, E. J. Dean, S. Fournel, D. Fong, M. C. Genovese, H. E. de Vegvar, K. Skriner, D. L. Hirschberg, R. I. Morris, S. Muller, G. J. Pruijn, W. J. van Venrooij, J. S. Smolen, P. O. Brown, L. Steinman, and P. J. Utz. 2002. Autoantigen microarrays for multiplex characterization of autoantibody responses. Nat Med 8:295.
12. Eberwine, J. 1996. Amplification of mRNA populations using aRNA generated from immobilized oligo(dT)-T7 primed cDNA. Biotechniques 20:584.
13. Cook, A. F., E. Vuocolo, and C. L. Brakel. 1988. Synthesis and hybridization of a series of biotinylated oligonucleotides. Nucleic Acids Res 16:4077.
14. Okamoto, T., T. Suzuki, and N. Yamamoto. 2000. Microarray fabrication with covalent attachment of DNA using bubble jet technology. Nat Biotechnol 18:438.
15. Fodor, S. P., J. L. Read, M. C. Pirrung, L. Stryer, A. T. Lu, and D. Solas. 1991. Light-directed, spatially addressable parallel chemical synthesis. Science 251:767.
16. Barone, A. D., J. E. Beecher, P. A. Bury, C. Chen, T. Doede, J. A. Fidanza, and G. H.

McGall. 2001. Photolithographic synthesis of high-density oligonucleotide probe arrays. Nucleosides Nucleotides Nucleic Acids 20:525.
17. Hubank, M., and D. G. Schatz. 1994. Identifying differences in mRNA expression by representational difference analysis of cDNA. Nucleic Acids Res 22:5640.
18. Lisitsyn, N., and M. Wigler. 1993. Cloning the differences between two complex genomes. Science 259:946.
19. Bussow, K., E. Nordhoff, C. Lubbert, H. Lehrach, and G. Walter. 2000. A human cDNA library for high-throughput protein expression screening. Genomics 65:1.
20. Altman, R., G. Alarcon, D. Appelrouth, D. Bloch, D. Borenstein, K. Brandt, C. Brown, T. D. Cooke, W. Daniel, D. Feldman, and et al. 1991. The American College of Rheumatology criteria for the classification and reporting of osteoarthritis of the hip. Arthritis Rheum 34:505.
21. Chomczynski, P., and N. Sacchi. 1987. Single-step method of RNA isolation by acid guanidinium thiocyanate-phenol-chloroform extraction. Anal Biochem 162:156.
22. Diatchenko, L., Y. F. Lau, A. P. Campbell, A. Chenchik, F. Moqadam, B. Huang, S. Lukyanov, K. Lukyanov, N. Gurskaya, E. D. Sverdlov, and P. D. Siebert. 1996. Suppression subtractive hybridization: a method for generating differentially regulated or tissue-specific cDNA probes and libraries. Proc Natl Acad Sci U S A 93:6025.
23. Gress, T. M., J. D. Hoheisel, G. G. Lennon, G. Zehetner, and H. Lehrach. 1992. Hybridization fingerprinting of high-density cDNA-library arrays with cDNA pools derived from whole tissues. Mamm Genome 3:609.
24. Lennon, G. G., and H. Lehrach. 1991. Hybridization analyses of arrayed cDNA libraries. Trends Genet 7:314.
25. Krenn, V., A. Konig, F. Hensel, C. Berek, M. M. Souto Cameiro, W. Haedicke, Y. Wang, H. Vollmers, and H. K. Muller-Hermelink. 1999. Molecular analysis of rheumatoid factor (RF)-negative B cell hybridomas from rheumatoid synovial tissue: evidence for an antigen-induced stimulation with selection of high mutated IgVH and low mutated IgVL/lambda genes. Clin Exp Immunol 11 5:168.
26. Leushner, J. 2001. MALDI TOF mass spectrometry: an emerging platform for genomics and diagnostics. Expert Rev Mol Diagn 1:11.
27. MacBeath, G., and S. L. Schreiber. 2000. Printing proteins as microarrays for high-throughput function determination. Science 289:1760.
28. Walter, G., K. Bussow, D. Cahill, A. Lueking, and H. Lehrach. 2000. Protein arrays for gene expression and molecular interaction screening. Curr Opin Microbiol 3:298.
29. Spiro, A., M. Lowe, and D. Brown. 2000. A bead-based method for multiplexed identification and quantitation of DNA sequences using flow cytometry. Appl Environ Microbiol 66:4258.
30. Nolan, J. P., and F. F. Mandy. 2001. Suspension array technology: new tools for gene and protein analysis. Cell Mol Biol (Noisy-le-grand) 47:1241.
31. Madersbacher, S., and P. Berger. 2000. Antibodies and immunoassays. Methods 21:41.
32. Valet, G., M. Valet, D. Tschope, H. Gabriel, G. Rothe, W. Kellermann, and H. Kahle. 1993. White cell and thrombocyte disorders. Standardized, self-leaming flow cytometric list mode data classification with the CLASSIF1 program system. Ann N Y Acad Sci 677:233.

1. Method for diagnosis and/or molecular definition and/or therapy development for chronic inflammatory joint diseases and other inflammatory, infectious or tumorous diseases in humans or animals, the method comprising, for humans, employment of substances which are sequences of single genes, a selection of genes or the entirety of the genes of Table 1 and/or of the genes coding for the proteins of Table 2 and/or employment of partial or complete sequences of single, a selection, or the entirety of proteins and peptides deduced from said gene sequences, and, for animals, employment of substances which are homologs of said substances for humans.

2. Method according to claim 1, in which the gene sequences their sequence are identical with or have a respective sequence identity of at least 80% in the protein-coding regions of, the genes of Table 1 or the genes coding for the proteins of Table 2.

3. Method according to claim 2, wherein the substances comprise sequence sections or partial sequences, which in respect to their sequence are identical with or which have a sequence identity of at least 80% with the respective sections of, the genes of Table 1 and the genes of claim 2.

4. Method according to any one of claims 1 to 3, further comprising
a High-Throughput method of (micro-) array-hybridisation or
a High-Throughput method using techniques of polymerase chain reaction for (semi-) quantification.

5. Method according to anv one of claims 1 to 3, further comprising using a labeled patient sample and a second, differently labeled control sample for a comparative double hybridisation to a an array together with the patient sample to effect a comparative red/green hybridisation.

6. Method according to claim 1, wherein said method is for diagnosis and the substances comprise partial or complete sequences of single, a selection, or the entirety of proteins or peptides deduced from said gene sequences.

7. Method according to claim 6, wherein the substances comprise single proteins, a selection of proteins or the entirety of the proteins of Table 2.

8. Method according to claim 6 or 7, wherein the protein or peptide sequences comprise partial sequences of proteins deduced form the genes of Table 1.

9. Method according to claim 6 or 7, wherein the substances in respect to their sequence are identical with or have a sequence identity of at least 80% with the proteins deduced from the genes of Table 1 or with the proteins of Table 2.

10. Method according to claim 6 or 7, further comprising. High-Throughput methods for analysis of protein expression comprising high definition, two-dimensional protein gel electrophoresis, MALDI techniques or
High-Throughput methods for protein spotting by means of protein arrays for screening for auto-antibodies for diagnosis of inflammatory joint diseases and other inflammatory, infectious or tumorous diseases in humans or
High-Throughput methods for protein spotting by means of protein arrays for screening for autoreactive T cells for diagnosis of inflammatory joint diseases and other inflammatory, infectious or tumorous diseases in humans or
Non-High-Throughput methods for protein spotting for screening for autoreactive T cells for diagnosis of inflammatory joint diseases and other inflammatory, infectious or tumorous diseases in humans.

11. Method according to claim 6 or 7, further comprising employment of antibodies which are specific for said partial or complete sequences of singles a selection or entirety of said proteins or peptides deduced from said gene sequences.

12. Method according to claim 1, for animals, wherein said substances are said homolois of said substances for humans.

13. Method according to claim 6 or 7, wherein mutations in said genes or alterations in the regulatory sequences of said genes are detected.

14. Method according to claim 6 or 7, wherein in said genes coding for the proteins of Table 2 or alterations in regulatory sequences of said genes are detected.

15. Method according to any one of claims 1 to 3, 6 and 7, wherein molecular definitions of said diseases in humans are determined.

16. Method according to any one of claims 1 to 3, wherein therapies for said for diseases are selected.

17. Method according to any one of claims 1 to 3, wherein progress of therapies for said diseases is monitored and the therapies are controlled based on said monitoring.

18. Method according to any one of claims 1 to 3, wherein therapy concepts are developed, said therapy concepts comprising direct or indirect impact on the expression of the said genes or gene sequences.

19. Method according to any one of claims 1 to 3, wherein therapy concepts are developed, said therapy concepts comprising direct or indirect impact on the expression of said proteins or partial protein sequences.

20. Method according to any one of claims 1 to 3, wherein therapy concepts are developed, said therapy concepts comprising direct or indirect impact on autoreactive T cells being directed against said proteins or partial protein sequences.

21. Method according to any one of claims 1 to 3, wherein biological action of the proteins deduced from said gene sequences is affected.

22. Method according to any one of claims 1 to 3, wherein direct molecular regulatory circuits/pathways, in which said genes and respective proteins deduced therefrom are involved, are affected.

23. Method according to any one of claims 1 to 3, further comprising constructing and applying interpretation algorithms.

24. Method according to any one of claims 1 to 3, further comprising developing biologically active drugs for said diseases.

25. A molecular tool comprised of an array, the array being comprised of different antibodies or molecules with a comparable protein-specific binding behaviour, the antibodies or molecules being capable of detecting the entirety of or a selection of the proteins deduced from the genes in Table 1 or the entirety of or a selection of said proteins of Table 2.

26. (canceled)

27. Method according to any one of claims 1 to 3, wherein said substances are employed in connection with analysis of blood samples or tissue samples in medical diagnosis.

28. Method according to any one of claims 1 to 3, wherein said substances are employed in connection with analysis of tissue samples in diagnosing and/or assessing the activity and/or developing a prognosis for and/or developing therapeutic options for said diseases.

29. Method according to any one of claims 1 to 3, wherein said substances are employed in connection with selection of therapies for said diseases.
